(19) United States
(12) Patent Application Publication
(10) Pub. No.: US 2026/0053760 A1
(43) Pub. Date: Feb. 26, 2026

(54) Compositions Suitable for Use on Skin Afflicted with Eczema

(71) Applicant: Kenvue Brands LLC, Summit, NJ (US)

(72) Inventor: Katie Spisak, Summit, NJ (US)

(21) Appl. No.: 19/105,494

(22) PCT Filed: Aug. 23, 2023
(86) PCT No.: PCT/IB2023/058385

§ 371 Date: Feb. 21, 2025

Related U.S. Application Data
(60) Provisional application No. 63/400,132, filed on Aug. 23, 2022.

(51) Int. Cl. A61K 31/191; A61K 9/00; A61K 9/06; A61K 31/192; A61K 31/194; A61K 47/10; A61K 47/14; A61K 47/26; A61K 47/32; A61K 47/44; A61K 47/46; A61P 17/00; A61P 17/04

(52) U.S. Cl. CPC A61K 31/191; A61K 9/0014; A61K 9/06; A61K 31/192; A61K 31/194; A61K 47/10; A61K 47/14; A61K 47/26; A61K 47/32; A61K 47/44; A61K 47/46; A61P 17/00; A61P 17/04

(57) ABSTRACT

Provided are composition suitable for skin afflicted with eczema comprising an alpha hydroxy acid; a volatile emollient; and a processed oat ingredient. Also provided are methods of using said compositions.

### FIELD

The present invention generally relates to compositions for suitable for use on skin afflicted with eczema. More specifically, the present invention relates to compositions comprising an alpha hydroxy acid, a volatile emollient and/or a processed oat ingredient.

### BACKGROUND

Atopic dermatitis, also known as eczema, is a chronic relapsing pruritic inflammation of the skin that can compromise quality of life. Atopic dermatitis affects 10-20% of children and 1-3% of adults worldwide with increasing prevalence in highly industrialized countries. Atopic dermatitis is characterized by an impaired epidermal barrier, dysregulation of innate and adaptive immunity, and a high susceptibility to bacterial colonization and infection. Hence, lubricants (ointments & creams) and topical or oral corticosteroids, which act on the immune system by blocking the production of substances that trigger allergic and inflammatory actions, remain the first-line treatments. Similarly, antihistamine can also be administrated. If pruritus does not respond to standard treatments, antibiotics might be considered.

Lubricants are effective in keeping the skin hydrated and repairing the skin barrier. However, the cosmetic acceptance of these types of formulations may be poor which is reflected in a lower compliance among the atopic dermatitis patients. Moreover, this approach is often not sufficient by itself. Corticosteroids on the other hand are powerful medications but are known to induce side effects, some of which might be severe in case of long term usage. While antihistamine can be used to treat itch associated with atopic dermatitis, they can cause sleepiness and may not help in all cases of atopic dermatitis. Finally, the use of antibiotics is controversial due to the raising occurrence of bacterial resistance.

Additionally, a treatment will ideally address the visual appearance of skin afflicted with eczema, and/or otherwise provide other sensorial benefits, such as a cooling sensation. However, traditional ingredients are often too harsh for use on the sensitive skin of individuals with eczema.

Accordingly, there exists a need for topical compositions which are suitable for use on eczematic skin and can address one or more of the concerns above.

### SUMMARY

One aspect of the invention pertains to a composition suitable for skin afflicted with eczema, the composition comprising:
a. an alpha hydroxy acid;
b. a volatile emollient; and
c. a processed oat ingredient.

In one or more embodiments, the alpha hydroxy acid is selected from the group consisting of glycolic acid, malic acid, tartaric acid, pyuric acid, mandelic acid, lactic acid, and combinations thereof. In some embodiments, the alpha hydroxy acid is present in an amount ranging from about 0.01 wt. % to about 5 wt. % by total weight of the composition. In one or more embodiments, the volatile emollient is selected from the group consisting of cyclopentasiloxane, dimethicone, isoparaffin cuts (C9-C16), ethyl trisiloxane, trisiloxane and caprylyl methicone, isododecane, and combinations thereof. In some embodiments, the volatile emollient is present in an amount ranging from about 0.01 wt. % to about 12 wt. % by total weight of the composition. In one or more embodiments, the processed oat ingredient is selected from the group consisting of oat extract, colloidal oatmeal, oat oil, fermented oat, avenanthramides, beta-glucan, modified oat grain material, and combinations thereof. In some embodiments, the processed oat ingredient is present in an amount ranging from about 0.01 wt. % to about 30 wt. % by total weight of the composition. In one or more embodiments, the composition further comprises a dermatologically-acceptable ingredient selected from the group consisting of viscosity increasers, humectants, skin conditioners, emulsifiers and combinations thereof. In some embodiments, the composition has a pH of from about 4.5 to about 5.5. In one or more embodiments, the composition is in the form of a lotion, gel or cream product.

Another aspect of the invention pertains to a composition suitable for skin afflicted with eczema, the composition comprising:
a. lactic acid;
b. isododecane; and
c. a processed oat ingredient selected from the group consisting of oat extract, colloidal oatmeal, oat oil and combinations thereof.

In one or more embodiments, the composition comprises:
a. from about 0.01 wt. % to about 3 wt. % lactic acid;
b. from about 0.01 wt. % to about 4 wt. % isododecane; and
c. from about 0.01 wt. % to about 6 wt. % combined of oat extract, colloidal oatmeal, and oat oil.

Another aspect of the invention pertains to a method of treating eczema, the method comprising topically applying to skin afflicted with eczema any of the compositions described herein. In one or more embodiments, the skin afflicted with eczema comprises mild to moderate eczema.

Another aspect of the invention pertains to a method of improving the appearance of skin afflicted with eczema, the method comprising topically applying to skin afflicted with eczema a composition comprising lactic acid. In one or more embodiments, the lactic acid is present in an amount ranging from about 0.01 wt. % to about 5 wt. % by total weight of the composition. In some embodiments, the skin afflicted with eczema comprises mild to moderate eczema. In one or more embodiments, the composition is applied to a lesional area. In some embodiments, the composition is applied to the face or body. In one or more embodiments, the composition is applied once or twice per day. In some embodiments, the lesional area exhibits a symptom selected from the group consisting of erythema, pruritus, exudation, excoriation, lichenification, dryness, tactile roughness, abnormal skin tone, burning, stinging, and combinations thereof. In one or more embodiments, the composition further comprises a volatile emollient. In some embodiments, the volatile emollient is selected from the group consisting of cyclopentasiloxane, dimethicone, isoparaffin cuts (C9-C16), ethyl trisiloxane, trisiloxane and caprylyl methicone, isododecane, and combinations thereof. In one or more embodiments, the volatile emollient is present in an amount ranging from about 0.01 wt. % to about 12 wt. % by total weight of the composition. In some embodiments, the composition further comprises a processed oat ingredient. In one or more embodiments, the processed oat ingredient is selected from the group consisting of oat extract, colloidal oatmeal, oat oil, fermented oat, avenanthramides, beta-glucan, modified oat grain material, and combinations thereof. In some embodiments, the processed oat ingredient is present in an amount ranging from about 0.01 wt. % to about 30 wt. % by total weight of the composition. In one or more embodiments, the composition further comprises a dermatologically-acceptable ingredient selected from the group consisting of thickeners, additional emollients, humectants, skin conditioners, emulsifiers and combinations thereof. In some embodiments, the composition is in the form of a lotion, gel or cream product.

Another aspect of the invention pertains to a method of providing a cooling sensation to skin afflicted with eczema, the method comprising topically applying to skin afflicted with eczema a composition comprising isododecane. In one or more embodiments, the isododecane is present in the composition in an amount ranging from about 0.01 wt. % to about 12 wt. % by total weight of the composition. In some embodiments, the skin afflicted with eczema comprises mild to moderate eczema. In one or more embodiments, the composition is applied to a lesional area. In some embodiments, the composition is applied to the face or body. In one or more embodiments, the composition is applied once or twice per day. In some embodiments, the lesional area exhibits a symptom selected from the group consisting of erythema, pruritus, exudation, excoriation, lichenification, dryness, tactile roughness, abnormal skin tone, burning, stinging, and combinations thereof. In one or more embodiments, the composition further comprises an alpha hydroxy acid. In some embodiments, the alpha hydroxy acid is present in an amount ranging from about 0.01 wt. % to about 5 wt. % by total weight of the composition. In one or more embodiments, the volatile emollient is selected from the group consisting of cyclopentasiloxane, dimethicone, isoparaffin cuts (C9-C16), ethyl trisiloxane, trisiloxane and caprylyl methicone, isododecane, and combinations thereof. In some embodiments, the composition further comprises a processed oat ingredient. In one or more embodiments, the processed oat ingredient is selected from the group consisting of oat extract, colloidal oatmeal, oat oil, fermented oat, avenanthramides, beta-glucan, modified oat grain material, and combinations thereof. In some embodiments, the processed oat ingredient is present in an amount ranging from about 0.01 wt. % to about 30 wt. % by total weight of the composition. In one or more embodiments, the cooling sensation persists for at least 5 minutes. In some embodiments, the composition is in the form of a lotion, gel or cream product.

### BRIEF DESCRIPTION OF THE FIGURES

FIG. 1 is a thermal image of skin at baseline;

FIG. 2 is a thermal image of the same skin as in FIG. 1 immediately post-treatment with a control and in accordance with one or more embodiments of the invention;

FIG. 3 is a thermal image of the same skin as in the preceding figures at 5 mins post-treatment;

FIG. 4 is a thermal image of the same skin as in the preceding figures at 10 mins post-treatment;

FIG. 5 is a thermal image of the same skin as in the preceding figures at 15 mins post-treatment;

FIG. 6 is a chart of qPCR copy number by cohort for S. Aureus;

FIG. 7 is a is a chart of qPCR copy number by cohort for S. Epidermidis;

FIG. 8 is a is a chart of average qPCR copy number by cohort for S. Epidermidis: S. Aureus;

FIG. 9 is a chart of genus level relative abundance of Staphylococcus to other genus;

FIG. 10 is a chart of species level relative abundance of S. Epidermidis, S. Aureus, and S. Hominis to other species;

FIG. 11 is a chart of alpha-diversity: genus level Chao1 richness;

FIG. 12 is a chart of alpha-diversity: genus level Shannon diversity;

FIG. 13 is a chart of alpha-diversity: genus level Simpson diversity;

FIG. 14 is a chart of alpha-diversity: species level Chao1 richness;

FIG. 15 is a chart of alpha-diversity: species level Shannon diversity;

FIG. 16 is a chart of alpha-diversity: species level Simpson diversity;

FIG. 17 is a chart of in vitro bacteria competition colloidal oat flour, oat extract, and glycerin; and

FIG. 18 is a chart of in vitro bacteria competition colloidal oat flour, oat extract, oat oil, lactic acid, and combination treatments.

### DETAILED DESCRIPTION

It is believed that one skilled in the art can, based on the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

Unless otherwise indicated, percentages used to express amounts of ingredients are percentage by weight (referred to as “weight %,” “wt %”, “% by weight” or “% (W/W)”). Similarly, weight ratios used to express relative proportions of ingredients are also determined using percentage by weight (i.e., weight ratios are calculated by dividing the percentage by weight of one ingredient by another). Unless stated otherwise, all ranges are inclusive of the endpoints, e.g., “from 4 to 9” includes the endpoints 4 and 9.

As used herein, “cosmetically acceptable” means that the ingredients the term describes are suitable for use in contact with tissues (e.g., the skin or hair) without undue toxicity, incompatibility, instability, irritation, allergic response, or the like.

As used herein, the term “safe and effective amount” means an amount sufficient to induce the desired effect, but low enough to avoid serious side effects. The safe and effective amount of the compound, extract, or composition will vary with, e.g., the age, health and environmental exposure of the end user, the duration and nature of the treatment, the specific extract, ingredient, or composition employed, the particular carrier utilized, and like factors.

As used herein, the term “about” refers to within 5% weight, within 4% weight, within 3% weight, within 2.5% weight, within 2% weight, or within 1% weight of a disclosed value.

One aspect of the invention pertains to composition suitable for skin afflicted with eczema, the composition comprising: (a) an alpha hydroxy acid; (b) a volatile emollient; and (c) a processed oat ingredient. It has been surprisingly discovered that such compositions are able to improve the cosmetic appearance of skin afflicted with eczema, skin barrier properties and hydration.

In particular, it has been surprisingly discovered that the alpha hydroxy acid, and particularly lactic acid, is well tolerated by skin afflicted with eczema. Accordingly, another aspect of the invention pertains to a method of improving the appearance of skin afflicted with eczema, the method comprising applying to skin afflicted with eczema a composition comprising lactic acid. As used herein, the term “improving the appearance of skin afflicted with eczema” refers to improvement in one or more of the visual symptoms of skin afflicted with eczema (e.g., dryness bumps, crusting scaliness, etc.), particularly in skin tone.

Additionally, it has been surprisingly discovered that the volatile emollient, and particularly isododecane, is sufficient to provide a cooling sensation gentle enough for the skin afflicted with eczema. Accordingly, another aspect of the invention pertains to a method of providing a cooling sensation to skin afflicted with eczema, the method comprising applying to skin afflicted with eczema a composition comprising isododecane.

### Alpha Hydroxy Acid

Alpha hydroxy acids are compounds which contain a carboxylic acid that is substituted with a hydroxyl group on the adjacent carbon atom. Examples of alpha hydroxy acids include, but are not limited to, glycolic acid, lactic acid, malic acid, tartaric acid, pyuric acid, mandelic acid, or any combination of any of the foregoing. In some embodiments, the alpha hydroxy acid comprises lactic acid. Suitability of the alpha hydroxy acid (especially lactic acid) is surprising, as such acids are generally thought of as being too harsh for skin afflicted with eczema.

The alpha hydroxy acid may be present in amounts ranging from about 0.01, 0.05, 0.1, 0.2, 0.3, 0.4, 0.5, 0.6, 0.7, 0.8, 0.9, 1.0, 1.5, 2, 2.5, 3, 3.5, 4, 4.5, or 5 to about 1.0, 1.5, 2, 2.5, 3, 3.5, 4, 4.5, or 5 wt. % of the total composition. In one or more embodiments, the alpha hydroxy acid is present in an amount ranging from about 0.01 wt. % to about 5 wt. % by total weight of the composition. In some embodiments, the alpha hydroxy acid is present in an amount ranging from about 0.1 wt. % to about 4 wt. % by total weight of the composition. In one or more embodiments, the alpha hydroxy acid is present in an amount ranging from about 0.5 wt. % to about 2 wt. % by total weight of the composition. In some embodiments, the alpha hydroxy acid is present in an amount ranging from about 0.5 wt. % to about 1.5 wt. % by total weight of the composition. In one or more embodiments, the alpha hydroxy acid is present in an amount of about 0.75 wt. % to about 1.5 wt. % by total weight of the composition. In some embodiments, the alpha hydroxy acid is present in an amount of about 1 wt. % to about 1.5 wt. % by total weight of the composition.

In one or more embodiments, the alpha hydroxy acid comprises lactic acid. The lactic acid may be present in amounts ranging from about 0.01, 0.05, 0.1, 0.2, 0.3, 0.4, 0.5, 0.6, 0.7, 0.8, 0.9, 1.0, 1.5, 2, 2.5, 3, 3.5, 4, 4.5, or 5 to about 1.0, 1.5, 2, 2.5, 3, 3.5, 4, 4.5, or 5 wt. % of the total composition. In one or more embodiments, the lactic acid is present in an amount ranging from about 0.01 wt. % to about 5 wt. % by total weight of the composition. In some embodiments, the lactic acid is present in an amount ranging from about 0.1 wt. % to about 4 wt. % by total weight of the composition. In one or more embodiments, the lactic acid is present in an amount ranging from about 0.5 wt. % to about 2 wt. % by total weight of the composition. In some embodiments, the lactic acid is present in an amount ranging from about 0.5 wt. % to about 1.5 wt. % by total weight of the composition. In one or more embodiments, the lactic acid is present in an amount of about 0.75 wt. % to about 1.5 wt. % by total weight of the composition. In some embodiments, the lactic acid is present in an amount of about 1 wt. % to about 1.5 wt. % by total weight of the composition.

### Volatile Emollient

As used herein, a “volatile emollient” refers to an emollient that has a flash point within 15° C. of the temperature of skin, and therefore do not persist on the skin after application. The volatile emollient may act as a cooling agent to provide a cooling sensation to the user's skin and will readily vaporize. Although not wishing to be bound to any particular theory, it is thought that the volatile emollient provides a cooling sensation on the skin solely due to evaporation of the ingredient from skin. It is thought there is a higher efficacy of cooling when it takes longer for ingredient to evaporate. According to one or more embodiments, the composition may include a volatile emollient, such as, isododecane. Other examples of volatile emollients may include cyclopentasiloxane, dimethicone, isoparaffin cuts (C9-C16), ethyl trisiloxane, trisiloxane and caprylyl methicone. In some embodiments, the volatile emollient comprises isododecane.

The volatile emollient may be present in amounts ranging from about 0.01, 0.05, 0.1, 0.2, 0.3, 0.4, 0.5, 0.6, 0.7, 0.8, 0.9, 1.0, 1.5, 2, 2.5, 3, 3.5, 4, 4.5, or 5 to about 5, 5.5, 6, 6.5, 7, 8, 9, 10, 11 or 12 wt. % of the total composition. In one or more embodiments, the volatile emollient is present in an amount ranging from about 0.1 wt. % to about 10 wt. % by total weight of the composition. In some embodiments, the volatile emollient is present in an amount ranging from about 0.1 wt. % to about 5 wt. % by total weight of the composition. In one or more embodiments, the volatile emollient is present in an amount ranging from about 0.5 wt. % to about 5 wt. % by total weight of the composition. In some embodiments, the volatile emollient is present in an amount ranging from about 0.5 wt. % to about 4 wt. % by total weight of the composition. In one or more embodiments, the volatile emollient is present in an amount ranging from about 0.5 wt. % to about 3 wt. % by total weight of the composition. In some embodiments, the volatile emollient is present in an amount ranging from about 1 wt. % to about 3 wt. % by total weight of the composition. In one or more embodiments, the volatile emollient is present in an amount of about 1.5 wt. % to about 2.5 wt. % by total weight of the composition. In some embodiments, the volatile emollient is present in an amount of about 1.75 wt. % to about 2.25 wt. % by total weight of the composition. In one or more embodiments, the volatile emollient is present in an amount of about 2 wt. % by total weight of the composition.

In one or more embodiments, the volatile emollient comprises isododecane. The isododecane may be present in amounts ranging from about 0.01, 0.05, 0.1, 0.2, 0.3, 0.4, 0.5, 0.6, 0.7, 0.8, 0.9, 1.0, 1.5, 2, 2.5, 3, 3.5, 4, 4.5, or 5 to about 5, 5.5, 6, 6.5, 7, 8, 9, 10, 11 or 12 wt. % of the total composition. In one or more embodiments, the isododecane is present in an amount ranging from about 0.1 wt. % to about 10 wt. % by total weight of the composition. In some embodiments, the isododecane is present in an amount ranging from about 0.1 wt. % to about 5 wt. % by total weight of the composition. In one or more embodiments, the isododecane is present in an amount ranging from about 0.5 wt. % to about 5 wt. % by total weight of the composition. In some embodiments, the isododecane is present in an amount ranging from about 0.5 wt. % to about 4 wt. % by total weight of the composition. In one or more embodiments, the isododecane is present in an amount ranging from about 0.5 wt. % to about 3 wt. % by total weight of the composition. In some embodiments, the isododecane is present in an amount ranging from about 1 wt. % to about 3 wt. % by total weight of the composition. In one or more embodiments, the isododecane is present in an amount of about 1.5 wt. % to about 2.5 wt. % by total weight of the composition. In some embodiments, the isododecane is present in an amount of about 1.75 wt. % to about 2.25 wt. % by total weight of the composition. In one or more embodiments, the isododecane is present in an amount of about 2 wt. % by total weight of the composition.

### Processed Oat Ingredient

As used herein, the term “processed oat ingredient” refers to an ingredient that is typically derived from a part of the oat plant (Avena sativa). Said ingredient can be either a processing (e.g., an extract, milling, fermenting) of one or more parts of the oat plant (e.g., grain, leaf, stem, seed) or can be a molecule found in the oat plant (e.g., beta-glucan, flavonoids, avenanthramides, lipids, peptides, etc.). The definition is intended to cover processed oat ingredients which are derived from other sources other than oat (e.g., from another plant or chemically synthesized), but are otherwise associated with oat. In one or more embodiments, the processed oat ingredient is selected from the group consisting of oat extract, colloidal oatmeal, oat flour, oat bran, oat protein, oat peptide, oat oil, fermented oat, avenanthramides, beta-glucan, modified oat grain material (e.g., chemically, enzymatically-, microorganism-modified), and combinations thereof. As used herein, “colloidal oatmeal” means the powder resulting from the grinding and further processing of whole oat grain meeting United States Standards for Number 1 or Number 2 oats. The colloidal oatmeal has a particle size distribution as follows: not more than 3 percent of the total particles exceed 150 micrometers in size and not more than 20 percent of the total particles exceed 75 micrometers in size. Examples of suitable colloidal oatmeals include, but are not limited to, “Tech-0” available from the Beacon Corporation and colloidal oatmeals available from Quaker. In one or more embodiments, the processed oat ingredient comprises oat extract, colloidal oatmeal, and oat oil. In some embodiments, the processed oat ingredient comprises oat extract. In one or more embodiments, the processed oat ingredient comprises colloidal oatmeal. In some embodiments, the processed oat ingredient comprises oat oil. In some embodiments, the processed oat ingredient is selected from the group consisting of oat extract, colloidal oatmeal, oat oil and combinations thereof. In some embodiments, the processed oat ingredient comprises oat extract, colloidal oatmeal, and oat oil. In one or more embodiments, the processed oat ingredient comprises avenanthramides. In some embodiments, the processed oat ingredient comprises fermented oat. In one or more embodiments, the processed oat ingredient comprises beta-glucan.

The processed oat ingredient(s) may be present in amounts ranging from about 0.01, 0.05, 0.1, 0.2, 0.3, 0.4, 0.5, 0.6, 0.7, 0.8, 0.9, 1.0, 1.5, 2, 2.5, 3, 3.5, 4, 4.5, or 5 to about 5, 5.5, 6, 6.5, 7, 8, 9, 10, 11 12, 15, 20, 25 or 30 wt. % of the total composition. In one or more embodiments, the processed oat ingredient(s) is present in an amount ranging from about 0.1 wt. % to about 10 wt. % by total weight of the composition. In some embodiments, the processed oat ingredient(s) is present in an amount ranging from about 0.1 wt. % to about 5 wt. % by total weight of the composition. In one or more embodiments, the processed oat ingredient(s) is present in an amount ranging from about 0.5 wt. % to about 5 wt. % by total weight of the composition. In some embodiments, the processed oat ingredient(s) is present in an amount ranging from about 0.5 wt. % to about 4 wt. % by total weight of the composition. In one or more embodiments, the processed oat ingredient(s) is present in an amount ranging from about 0.5 wt. % to about 3 wt. % by total weight of the composition. In some embodiments, the processed oat ingredient(s) is present in an amount ranging from about 1 wt. % to about 3 wt. % by total weight of the composition. In one or more embodiments, the processed oat ingredient(s) is present in an amount of about 1 wt. % to about 2 wt. % by total weight of the composition. In some embodiments, the processed oat ingredient(s) is present in an amount of about 1 wt. % by total weight of the composition. In one or more embodiments, the processed oat ingredient(s) is present in an amount of about 2 wt. % by total weight of the composition.

In one or more embodiments, the processed oat ingredient comprises colloidal oatmeal, and the colloidal oatmeal is present in an amount of about 0.5 wt. % to about 3 wt. % by total weight of the composition. In some embodiments, the colloidal oatmeal is present in an amount of about 1 wt. % to about 3 wt. % by total weight of the composition. In one or more embodiments, the colloidal oatmeal is present in an amount of about 1 wt. % to about 2 wt. % by total weight of the composition. In some embodiments, the colloidal oatmeal is present in an amount of about 1 wt. % by total weight of the composition. In one or more embodiments, the colloidal oatmeal is present in an amount of about 2 wt. % by total weight of the composition.

In some embodiments, the processed oat ingredient comprises oat extract, colloidal oatmeal, and oat oil, and the total amount of the three ingredients may be present in amounts ranging from about 0.01, 0.05, 0.1, 0.2, 0.3, 0.4, 0.5, 0.6, 0.7, 0.8, 0.9, 1.0, 1.5, 2, 2.5, 3, 3.5, 4, 4.5, or 5 to about 5, 5.5, 6, 6.5, 7, 8, 9, 10, 11 12, 15, 20, 25 or 30 wt. % of the total composition. In one or more embodiments, the total amount of the oat extract, colloidal oatmeal, and oat oil is from about 0.1 wt. % to about 10 wt. % by total weight of the composition. In some embodiments, the total amount of the oat extract, colloidal oatmeal, and oat oil is about 0.1 wt. % to about 5 wt. % by total weight of the composition. In one or more embodiments, the total amount of the oat extract, colloidal oatmeal, and oat oil is about 0.5 wt. % to about 5 wt. % by total weight of the composition. In some embodiments, the total amount of the oat extract, colloidal oatmeal, and oat oil is about 0.5 wt. % to about 4 wt. % by total weight of the composition.

### Other Ingredients

Any of the compositions described herein may further comprise any of a variety of additional other auxiliary ingredients used conventionally in healthcare/personal care compositions (referred to generally as “personal care components”). These other personal care components nonexclusively include one or more, pearlescent or opacifying agents, conditioners, humectants, chelating agents, actives, exfoliants, and additives which enhance the appearance, feel and fragrance of the compositions, such as colorants, fragrances, preservatives, pH adjusting agents, rheology modifiers, and the like. Such auxiliary ingredients, when present, will be cosmetically/dermatologically acceptable and present in safe and effective amounts. In one or more embodiments, any of the compositions described herein may further comprise one or more dermatologically-acceptable ingredients selected from the group consisting of thickeners, humectants, skin conditioners, additional emollients (i.e., other than volatile emollients), emulsifiers and combinations thereof.

Any of a variety of commercially available humectants, are suitable for use in the present invention. What is meant by a humectant is a compound intended to increase the water content of the top layers of skin (e.g., hygroscopic compounds). Examples of suitable humectants include those found in Chapter 35, pages 399-415 (Skin Feel Agents, by G Zocchi) in Handbook of Cosmetic Science and Technology (edited by A. Barel, M. Paye and H. Maibach, Published in 2001 by Marcel Dekker, Inc New York, NY) and include, but are not limited to, glycerin, sorbitol or trehalose (e.g., α,α-trehalose, β,β-trehalose, α,β-trehalose) or a salt or ester thereof (e.g., trehalose 6-phosphate).

When present, the humectant may be present in an amount of from about 1% to about 30%, about 1 to about 20%, about 2% to about 15%, about 5% to about 15%, about 10% to about 15%, about 12% to about 15%, about 13% to about 15%, or about 14% by weight % of the total composition. In one or more embodiments, the humectant comprises glycerin and is present in an amount of from about 1% to about 30%, about 1 to about 20%, about 2% to about 15%, about 5% to about 15%, about 10% to about 15%, about 12% to about 15%, about 13% to about 15%, or about 14% by weight % of the total composition.

Any of a variety of skin conditioners are suitable for use in this invention. Examples include: glycols (e.g., caprylyl glycol), cationic surfactants (e.g. Cetrimonium Chloride, Stearamidopropyl Dimethylamine, Distearyldimonium Chloride, Lauryl Methyl Gluceth-10 Hydroxypropyldimonium Chloride); cationic polymers (e.g. cationically-modified polysaccharides, including Polyquaternium-10, Polyquaternium-24, Polyquaternium-67, Starch Hydroxypropyltrimonium Chloride, Guar Hydroxypropyltrimonium Chloride, and Hydroxypropyl Guar Hydroxypropyltrimonium Chloride, and cationic polymers derived from the (co)polymerization of ethylenically-unsaturated cationic monomers with optional hydrophilic monomers, including Polyquaternium-5, Polyquaternium-6, Polyquaternium-7, Polyquaternium-11, Polyquaternium-14, Polyquaternium-15, Polyquaternium-28, Polyquaternium-39, Polyquaternium-44; Polyquaternium-76); silicones and silicone derivatives (e.g. Dimethicone and derivatives thereof, such as alkyl-, polyalkylencoxy-, cationically-, anionically-modified dimethicone (co)polymers); and emollients (e.g. Caprylic/Capric Triglycerides, Petrolatum, Di-PPG-2 Myreth-10 Adipate).

Additional emollients include compounds that help to maintain the soft, smooth, and pliable appearance of the skin (e.g., by remaining on the skin surface or in the stratum corneum to act as a lubricant). Examples of suitable emollients include those found in Chapter 35, pages 399-415 (Skin Feel Agents, by G Zocchi) in Handbook of Cosmetic Science and Technology (edited by A. Barel, M. Paye and H. Maibach, Published in 2001 by Marcel Dekker, Inc New York, N.Y.), and include, but are not limited to, esters (e.g., isopropyl palmitate), petrolatum, hexyldecyl stearate and plant, nut, and vegetable oils and butters such as Butyrospermum parkii (shea) butter, Euphorbia cerifera (candelilla) wax; hydrogenated vegetable oil, macadamia nut oil, rice bran oil, grape seed oil, palm oil, prim rose oil, hydrogenated peanut oil, and avocado oil.

Compositions useful in the present invention may also include any of a variety of conventional thickening agents. Examples of such thickening agents include: carbomers (e.g., CARBOPOL ULTREZ 30 polymer), cetyl alcohol, electrolytes (e.g. Sodium Chloride, Ammonium Chloride, Magnesium Chloride); naturally-derived polysaccharides (e.g. Xanthan Gum, Dehydroxanthan Gum, Cyamopsis tetragonoloba (Guar) Gum, Cassia Gum, Chondrus crispus (Carrageenan) Gum, Alginic Acid and alginate gums (Algin, Calcium Alginate, etc.), Gellan Gum, Pectin, Microcrystalline Cellulose); derivatives of natural polysaccharides (e.g. Hydroxyethylcellulose, Ethyl Hydroxyethylcellulose, Cetyl Hydroxyethylcellulose, Methylcellulose, Hydroxypropylcellulose, Sodium Carboxymethylcellulose, Hydroxypropyl Methylcellulose, Hydroxypropyl Guar, Carboxymethyl Hydroxypropyl Guar, C18-22 Hydroxylalkyl Hydroxypropyl Guar); alkali-swellable emulsion (ASE) polymers (e.g. Acrylates Copolymer, available under the trade name Carbopol® AQUA SF-1 from Noveon Consumer Specialties, Brecksville, Ohio, and Acrylates Copolymer available under the trade name Aculyn™ 33 from Dow Personal Care, Spring House, Pa.); hydrophobically-modified alkali-swellable emulsion (HASE) polymers (e.g. Acrylates/Steareth-20 Methacrylate Copolymer, Acrylates/Steareth-20 Methacrylate Crosspolymer, and Acrylates/Ceteth-20 Itaconate Copolymer); hydrophobically-modified acid-swellable emulsion polymers (e.g. Acrylates/Aminoacrylates/C10-30 Alkyl PEG-20 Itaconate Copolymer and Polyacrylate-1 Crosspolymer); hydrophobically-modified acrylate crosspolymers, such as Acrylates C10-30 Alkyl Acrylates Crosspolymer, available under the trade name Carbopol® 1382 from Lubrizol Corp., Brecksville, Ohio; sodium polyacrylate (available as COSMEDIA SP), and hydrophobic non-ethoxylated micellar thickeners (e.g. Glyceryl Oleate, Cocamide MIPA, Lauryl Lactyl Lactate, or Sorbitan Sesquicaprylate).

The compositions described herein may contain additional surfactants/emulsifiers, including anionic, cationic, nonionic and amphoteric surfactants. Suitable emulsifiers include olive-derived emulsifiers, such as olivates. For example, one suitable emulsifier includes Olivem 1000 (sold by Hallstar), which is a combination of cetearyl olivate and sorbitan olivate. Examples of suitable nonionic surfactants include, but are not limited to the fatty alcohol acid or amide ethoxylates, monoglyceride ethoxylates, sorbitan ester ethoxylates alkyl polyglycosides, and mixtures thereof. One suitable nonionic surfactant is the polyoxyethylene derivatives of polyol esters, wherein the polyoxyethylene derivative of polyol ester (1) is derived from (a) a fatty acid containing from about 8 to about 22, and preferably from about 10 to about 14 carbon atoms, and (b) a polyol selected from sorbitol, sorbitan, glucose, α-methyl glucoside, polyglucose having an average of about 1 to about 3 glucose residues per molecule, glycerin, pentaerythritol and mixtures thereof, (2) contains an average of from about 10 to about 120, and preferably about 20 to about 80 oxyethylene units; and (3) has an average of about 1 to about 3 fatty acid residues per mole of polyoxyethylene derivative of polyol ester.

Examples of polyoxyethylene derivatives of polyol esters include, but are not limited to PEG-80, sorbitan laurate, and polysorbate 20. PEG-80 sorbitan laurate, which is a sorbitan monoester of lauric acid ethoxylated with an average of about 80 moles of ethylene oxide, is available commercially from ICI Surfactants of Wilmington, Del. under the tradename, “Atlas G-4280.” Polysorbate 20, which is the laurate monoester of a mixture of sorbitol and sorbitol anhydrides condensed with approximately 20 moles of ethylene oxide, is available commercially from ICI Surfactants of Wilmington, Del. under the tradename “Tween 20.”

Another class of suitable nonionic surfactants includes long chain alkyl glucosides or polyglucosides, which are the condensation products of (a) a long chain alcohol containing from about 6 to about 22, and preferably from about 8 to about 14 carbon atoms, with (b) glucose or a glucose-containing polymer. The alkyl gluocosides have about 1 to about 6 glucose residues per molecule of alkyl glucoside. A preferred glucoside is decyl glucoside, which is the condensation product of decyl alcohol with a glucose polymer and is available commercially from Henkel Corporation of Hoboken, N.J. under the tradename, “Plantaren 2000.”

The compositions of the present invention may also contain an amphoteric surfactant. As used herein, the term “amphoteric” shall mean: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. The amphoteric surfactants are disclosed herein without a counter ion. One skilled in the art would readily recognize that under the pH conditions of the compositions of the present invention, the amphoteric surfactants are either electrically neutral by virtue of having balanced positive and negative charges, or they have counter ions such as alkali metal, alkaline earth, or ammonium counter ions. Examples of amphoteric surfactants suitable for use in the present invention include, but are not limited to, amphocarboxylates such as alkylamphoacetates (mono or di); alkyl betaines; alkylamidoalkyl betaines; alkylamidoalkyl sultaines; alkylamphophosphates; phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures thereof.

Classes of cationic surfactants that are suitable for use in this invention include alkyl quaternaries (mono, di, or tri), benzyl quaternaries, ester quaternaries, ethoxylated quaternaries, alkyl amines, and mixtures thereof, wherein the alkyl group has from about 6 carbon atoms to about 30 carbon atoms, with about 8 to about 22 carbon atoms being preferred. These cationic surfactants can be employed in composition of the present invention in an amount, based upon the total weight of the composition, from about 0.01% to about 18%, or from about 0.05% to about 15% or from about 0.1% to about 10%.

Examples of suitable chelating agents include those which are capable of protecting and preserving the compositions of this invention. The chelating agent may include ethylenediamine tetracetic acid (“EDTA”), such as tetrasodium EDTA, available commercially from Dow Chemical Company of Midland, Mich. under the tradename, “Versene 100XL” and is present in an amount, based upon the total weight of the composition, from about 0 (if unused) to about 0.5 percent or from about 0.05 percent to about 0.25 percent.

Suitable preservatives include, for example, parabens, quaternary ammonium species, phenoxyethanol, benzoates, sorbates, DMDM hydantoin, and are present in the composition in an amount, based upon the total weight of the composition, from about 0 (if unused) to about 1 percent or from about 0.05 percent to about 0.5 percent. The preservatives are preferably selected from the group consisting of potassium benzoate, potassium sorbate and combinations thereof. The preservatives may also be combined with other ingredients which provide a booster effect. One example of such a booster is caprylyl glyceryl ether, which is an emollient which can provide a boosting effect to potassium benzoate and/or potassium sorbate. The ratio of preservative to caprylyl glyceryl ether may be from about 0.3 to 0.9, preferably 0.6.

The pH of the composition may be adjusted using pH adjusters commonly used in the art. In one or more embodiments, the composition has a pH of from about 4.5 to about 5.5. In some embodiments, the pH of the composition ranges from about 3, 3.1, 3.2, 3.3, 3.4, 3.5, 3.6, 3.7, 3.8 or 3.9 to about 3.8, 3.9, 4, 4.1, 4.2, 4.3, 4.4, 4.5, 4.6, 4.7, 4.8, 4.9 or 5. In some embodiments, the pH of the composition ranges from about 3 to about 5, or about 3.5 to about 4.5.

### EXEMPLARY EMBODIMENTS

One exemplary composition suitable for skin afflicted with eczema according to one or more embodiments, comprises:
a. lactic acid;
b. isododecane; and
c. a processed oat ingredient selected from the group consisting of oat extract, colloidal oatmeal, oat oil and combinations thereof.

In further embodiments, the composition comprises:
a. from about 0.01 wt. % to about 3 wt. % lactic acid;
b. from about 0.01 wt. % to about 4 wt. % isododecane; and
c. from about 0.01 wt. % to about 6 wt. % combined of oat extract, colloidal oatmeal, and oat oil.

### Product Form and Packaging

A variety of product forms and packaging may be suitable for any of the compositions described herein. As used herein, a “product” is optionally in finished packaged form. In one embodiment, the package is a container such as a plastic, metal or glass tube or jar containing the composition. In one or more embodiments, the composition may be squeezed or pumped out of the container. The product may further contain additional packaging such as a plastic or cardboard box for storing such container. In one or more embodiments, the product includes a composition of the invention and contains instructions directing the user to apply the composition to the skin.

Any of the compositions may be in the form of emulsions, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. Reference in the instant application is made to two types of viscosity measurements: A) “rheometer viscosity,” which is measured as a steady-state value at an applied shear rate of 20 s⁻¹ in a rheometer, at 25° C., and B) “Brookfield viscosity,” which is measured at 5 or 10 RPM after one minute at 25° C. in 4 oz jar using spindle RV #4 or RV #5. Unless otherwise specified, the viscosity referenced is the rheometer viscosity.

In one or more embodiments, the compositions described herein may be in the form of a lotion, gel or cream product. Lotion, gel and cream products may advantageously have the properties to allow for the compositions to be flowable and applied onto skin. Such products are generally thin enough to allow the volatile emollient to evaporate and provide the cooling effect. If too thick, as with an ointment, occlusive or patch, the user may not perceive a cooling affect.

In one or more embodiments, the compositions described herein are in the form of a lotion. As used herein, the term “lotion” means a predominantly water-containing topical preparation of light texture and fresh watery sensation, having a viscosity of from about 1,000 cPs to 4,000 cPs. Lotions or serums typically contain at least one emollient in addition to a solvent. Lotions/serums may comprise from about 1% to about 20% (e.g., from about 5% to about 10%) of an emollient(s) and from about 50% to about 90% (e.g., from about 60% to about 80%) of water.

In one or more embodiments, the compositions described herein are in the form of a cream. As used herein, the term “cream” means a predominantly water-containing topical preparation of rich texture having a viscosity of from about 2,000 cPs to 8,000 cPs. A cream typically contains from about 5% to about 50% (e.g., from about 10% to about 20%) of an emollient(s) and from about 45% to about 85% (e.g., from about 50% to about 75%) of water.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type, and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel. As used herein, the term “gel” means a predominantly water-containing topical preparation of rich texture containing dispersing polymer and exhibiting yield value of about 0.1 Pa or more. The gel may contain a gelling agent. Such gels typically contain between about 0.1% and 5%, by weight, of such gelling agents.

The compositions of the present invention can also be formulated into a solid formulation (e.g., wipe). The composition of the present invention can also be combined with and/or impregnated on to a solid, semi-solid, or dissolvable substrate (e.g., a wipe, mask, pad, glove, or strip).

The compositions described herein can be provided to the consumer in a container, e.g., a bottle, tube, etc. Individual packettes enclosing measured portions of the composition may also be used. To dispense the composition from a bottle, a pump, squeezable valve, or a removable screw cap may be used.

### Methods

Various methods described herein pertain to topically applying one or more of the compositions described to skin affected by eczema. As described above, the compositions may be used for treating skin afflicted with eczema, for improving the appearance of skin afflicted with eczema, and/or providing a cooling sensation to skin afflicted with eczema.

Any suitable method of topically applying the compositions described herein to the skin in need may be used. As used herein, “topically applying” means directly laying on or spreading on outer skin, by use of the hands or an applicator such as a wipe, roller, or spray. For example, the composition may be applied directly from a package to the skin in need, by hand to the skin in need, or may be transferred from a substrate such as a wipe or mask, or a combination of two or more thereof. In other embodiments, the composition may be applied via a dropper, tube, roller, spray, or added to a bath or otherwise to water to be applied to the skin, and the like. Such topical application may be to any skin in need of treatment on the body, for example skin of the face, lips, neck, chest, back, buttocks, arms, axilla, and/or legs.

The skin afflicted with eczema may comprise mild to moderate eczema. The compositions described herein may be applied to a lesional area on the skin afflicted with eczema. The lesional area may be one exhibiting a symptom selected from the group consisting of erythema, pruritus, exudation, excoriation, lichenification, dryness, tactile roughness, abnormal skin tone, burning, stinging, and combinations thereof. In some embodiments, the composition is applied to the face or body. The compositions may be applied to the skin as needed, or otherwise how often the user desires. In one or more embodiments, the composition is applied once or twice per day.

### EXAMPLES

### Example 1: Preparation of a Composition

Test composition was prepared by first adding water to a vessel. The colloidal oat flour was then stirred into the water with continuous mixing followed by the lactic acid and the carbomer, with ensuring homogenous mixture after each ingredient was added. The mixture was heated to 75-80° C. while at the same time adding the caprylyl glycol until uniform. Meanwhile half of the glycerin and sodium polyacrylate were pre-mixed together until homogeneous, then added to the main vessel at 75-80° C. Next, each of the following materials were added to the vessel with continuous mixing and maintaining the temperature at 75-80° C.: ceteryl olivate, sorbitan olivate (Olivem 1000), cetyl alcohol, Euphorbia cerifera (Candelilla) wax, hydrogenated vegetable oil, vegetable oil (Cegesoft VP), Butyrospermum parkii (Shea) butter, isopropyl palmitate, isododecane, and Avena sativa (Oat) Kernel oil. Continuous mixing continued until the batch was uniform, then the temperature was cooled to 68-75° C. for a minimum of 5 minutes. The temperature was then allowed to begin cooling while adding the other half of the glycerin. Once the batch had cooled to at or below 40° C. the following materials were added with continuous mixing until uniform: water, glycerin, Avena sativa (Oat) Kernel extract (Dragocalm), 1,2 hexanediol, caprylyl glycol, and tropolone (Symdiol 68 T). Once the mixture cooled at or below 35° C. the pH was tested and adjusted to 4.8 with 50% w/w sodium hydroxide in water. Lastly, the mixture was homogenized and transferred into containers for storage. See Table 1 below for final composition.

TABLE 1

| Tradename | INCI | | % in formulation |
|---|---|---|---|
| Permethyl 99/ | Isododecane | Volatile Emollient | 2 |
| Isododecane¹ | | | |
| Glycerin 99.7 Percent | Glycerin | Humectant | 14 |
| Min USP Kosher² | | | |
| Oat Lipid E³ | Avena Sativa (Oat) | Processed Oat | 0.5 |
| | Kernel Oil | Ingredient | |
| DRAGOCALM | Water; Glycerin; | Processed Oat | 1.0 |
| 674463⁴ | Avena Sativa (Oat) | Ingredient | |
| | Kernel Extract | | |
| Colloidal Oat Flour⁵ | Colloidal Oatmeal | Processed Oat | 2.0 |
| | | Ingredient | |
| PURAC PF 90⁶ | Lactic Acid | Alpha hydroxy acid | 1.11 |
| Radia 7732 RSPO | Isopropyl Palmitate, | Emollients | 6.25 |
| MB (Formerly | Euphorbia Cerifera | | |
| Known as RADIA | (Candelilla) Wax; | | |
| 7732)⁷, Cegesoft VP⁸ | Hydrogenated | | |
| | Vegetable Oil; | | |
| | Vegetable Oil | | |
| Hydrolite CG⁹, | Caprylyl Glycol, 1,2- | Skin Conditioners | 4.01 |
| 177441 Symdiol | Hexanediol; Caprylyl | | |
| 68T¹⁰, Cetiol SB 45¹¹ | Glycol; Tropolone, | | |
| | Butyrospermum | | |
| | Parkii (Shea) Butter | | |
| Carbopol Ultrez 30 | Carbomer, Cetyl | Emulsifiers & | 2.55 |
| Polymer¹², | alcohol, Cetearyl | Viscosity Increasers | |
| VEGAROL 1698¹³ | Olivate; Sorbitan | | |
| Olivem 1000¹⁴ | Olivate, Sodium | | |
| Cosmedia SP¹⁵ | Polyacrylate | | |
| Sodium Hydroxide, | Sodium Hydroxide | pH Adjuster | 1 |
| Pellets N.F.-F.C.C¹⁶ | | | |
| Purified Water | Water | Solvent | 65.58 |

¹100% Isododecane
²100% Glycerin
³100% Avena Sativa (Oat) Kernel Oil
⁴0.55% Avena Sativa Kernal Extract, 49.625% Glycerin, 49.625% water, 0.2 potassium sorbate
⁵100% Colloidal Oatmeal
⁶90% Lactic acid, 10% water
⁷100% Isopropyl Palmitate
⁸3% Euphorbia Cerifera (Candelilla) Wax, 10% Hydrogenated Vegetable Oil; 87% Vegetable Oil
⁹100% Caprylyl Glycol
¹⁰49.5% 1,2-Hexanediol, 49.5% Caprylyl Glycol, 1% Tropolone
¹¹100% Butyrospermum Parkii (Shea) Butter
¹²100% Carbomer
¹³100% Cetyl alcohol
¹⁴60% Ceteryl olivate, 40% Sorbitan olivate
¹⁵89% Sodium Polyacrylate, 11% water
¹⁶100% Sodium hydroxide

### Example 2: Clinical Study

A single-center, monadic clinical study was conducted to evaluate the efficacy and perceived benefits in itch and tolerability of a test composition when used on an eczema target lesion of subjects with mild to moderate eczema. The study was run by a Principal investigator, who was a board certified dermatologist.

The study was inclusive of a variety of demographics including age, sex, ethnicity, race, skin type, and skin tone. Exclusion criteria included having a skin condition that may interfere with the outcomes of the study such as, severe eczema, psoriasis, rosacea, rashes, many and/or severe excoriations, etc.; using Class I-III topical corticosteroids or systemic medications that may have a direct or indirect effect on eczema; or having known allergies or adverse reactions to common skin care ingredients. Enrollment criteria included subjects having mild to moderate eczema severity rating (Rajka and Langeland Severity Index) at visit 1, having at least one targeted eczema lesional area at visit 1 with a score of 5-12 on the Atopic Dermatitis Severity Index (ADSI), with mild or moderate, and erythema. 35 subjects completed study participation.

Subjects were also instructed to wash and remove all topical products on eczema lesion area at least 2-3 hours prior to each visit for evaluations. During the course of the study, subjects applied the test composition on the selected target lesion at least twice per day (once in the morning and once in the evening), as directed. Subjects performed the first application of the test composition on the target lesion on-site after completion of baseline evaluations.

Clinical evaluations were conducted at visit 1 (baseline), visit 2 (day 3), visit 3 (week 1/day 7), and visit 4 (week 2/day 14). Principal investigator (PI) evaluated the efficacy of the test composition by grading the various endpoints discussed in the subjects' eczema lesions using Atopic Dermatitis Severity Index (ADSI). The reported ADSI score was the sum of the individual scores. Additionally, the subject's target lesion was evaluated for dryness, tactile roughness, and skin tone of lesion compared to normal overall skin tone. Lastly, the PI assessed tolerance of subject's target lesion for overall irritation and asked each subject to rate the degree of burning/stinging sensation on the target eczema lesion. See Tables 2-6 for scoring criteria.

TABLE 2
Rajka and Langeland Severity Index Assessment

| | Score ^{a} |
|---|---|
| I - Extent | |
| (a) Childhood and Adult Phase | |
| Less than approximately 9% of the body area | 1 |
| Involvement evaluated to be more than score 1, | 2 |
| less than score 3 | |
| More than approximately 36% of the body area involved | 3 |
| (b) Infantile Phase | |
| Less than approximately 18% of the skin involved | 1 |
| Involvement evaluated to be more than score 1, | 2 |
| less than score 3 | |
| More than approximately 54% of the skin involved | 3 |
| II - Course | |
| More than 3 months of remission during a year ^{b} | 1 |
| Less than 3 months remission during a year ^{b} | 2 |
| Continuous course | 3 |
| III - Intensity | |
| Mild itch, only exceptionally disturbing night's sleep | 1 |
| Itch, evaluated to be more than score 1, less than score 3 | 2 |
| Severe itch, usually disturbing night's sleep | 3 |

^{a} When in doubt, a half-score of 1.5 or 2.5 may be used.
^{b} Criteria adapted to the actual period of the disease as per Physician's assessment.

TABLE 3
Rajka and langeland Severity Index Assessment (continued)
“Rule of Nine” division of the body surface
to estimate the extend of dermatitis

| Body Site | Percentage |
|---|---|
| Head | 9 |
| Upper extremity (score both arms separately) | 9 |
| Body, anterior | 18 |
| Body, posterior | 18 |
| Lower extremity (score both legs separately) | 18 |
| Genital area | 1 |

TABLE 4
Atopic Dermatitis Severity Index (ADSI) Assessment
Erythema (redness present specifically in the target lesion)

| | |
|---|---|
| 0 = None | No redness |
| 1 = Mild | Mildly detectable erythema; pink |
| 2 = Moderate | Dull red; clearly distinguishable |
| 3 = Severe | Deep, dark red; marked and extensive |

Pruritus (itching; present specifically in the target lesion)

| | |
|---|---|
| 0 = None | No itching |
| 1 = Mild | Occasional, slight itching |
| 2 = Moderate | Frequent or intermittent itching; |
| | does not disturb sleep |
| 3 = Severe | Constant, bothersome itching that |
| | disturbs sleep or normal activity |

Exudation (oozing or crusting of the target lesion)

| | |
|---|---|
| 0 = None | No oozing or crusting |
| 1 = Mild | Minor or faint signs of oozing or crusting |
| 2 = Moderate | Definite oozing or crusting present |
| 3 = Severe | Marked and extensive oozing or crusting present |

Excoriation (evidence of scratching in the target lesion)

| | |
|---|---|
| 0 = None | No evidence of excoriation |
| 1 = Mild | Mild excoriation present |
| 2 = Moderate | Definite excoriation present |
| 3 = Severe | Marked, deep, or extensive excoriation present |

Lichenification (epidermal thickening of the target lesion)

| | |
|---|---|
| 0 = None | No epidermal thickening |
| 1 = Mild | Minor epidermal thickening |
| 2 = Moderate | Moderate epidermal thickening; |
| | accentuated skin lines |
| 3 = Severe | Severe epidermal thickening; |
| | deeply accentuated skin lines |

TABLE 5
Scoring for Clinical Evaluations
Dryness

| | |
|---|---|
| 0 = None | No dryness |
| 1 = Mild | Slight, but definite dryness |
| 2 = Moderate | Definite dryness |
| 3 = Severe | Marked dryness |

Tactile roughness

| | |
|---|---|
| 0 = None | Skin perfectly smooth |
| 1 = Mild | Mild roughness |
| 2 = Moderate | Moderate roughness |
| 3 = Severe | Severe roughness |

Skin tone of lesion compared to normal overall skin tone

| | |
|---|---|
| 0 = None | Same overall tone (color) |
| 1-3 = Mild | Slightly different color |
| 4-6 = Moderate | Moderately different color |
| 7-9 = Severe | Marked, severe difference in color |

TABLE 6
Scoring for Tolerance Evaluations (Clinical and Subject)

Overall irritation
0 = None
1 = Mild
2 = Moderate
3 = Severe
4 = Very severe

Burning/Stinging

| | |
|---|---|
| 0 = None | No burning/stinging of the treatment area |
| 1 = Mild | Slight burning/stinging sensation of the treatment area |
| 2 = Moderate | Definite burning/stinging of the treatment area |
| 3 = Severe | Intense burning/stinging sensation of the treatment area |

The mean scores for each assessment were reported in Table XX below.

TABLE 7

Mean Scores

| | | Day | Week | Week |
|---|---|---|---|---|
| Efficacy measure | Baseline | 3 | 1 | 2 |
| Dryness | 2.14 | 0.66* | 0.51* | 0.51* |
| Tactile | 1.91 | 0.66* | 0.49* | 0.46* |
| Roughness | | | | |
| Skin Tone (Target | 3.8 | 2.86* | 1.40* | 1.20* |
| area vs. | | | | |
| Surrounding | | | | |
| Skin) | | | | |
| Pruritus (from | 2.11 | 0.94* | 0.57* | 0.37* |
| ADSI) | | | | |
| ADSI | 6.11 | 3.14* | 1.89* | 1.63* |
| Overall Lesion | 1.89 | 0.66* | 0.11* | 0.43* |
| Irritation | | | | |
| Burning/Stinging | 1.69 | 0.43* | 0.20* | 0.11* |

*Statistically significant improvement in all measures, at all timepoints (p < 0.05) versus baseline

Overall results from study shown in Table 7 indicate that the test composition was effective in improving the cosmetic appearance of target eczema lesion, skin barrier properties, and skin hydration, when used over the course of 2 weeks by subjects with mild to moderate eczema. The performance of the test composition in this study was surprising for a composition containing lactic acid. Dermatologists generally would not recommend lactic acid for use on eczema skin due to the irritating nature of the ingredient. However, the high tolerability of test composition including lactic acid on eczema skin as shown here, in particular for burning/stinging, puritis and irritation is especially surprising.

### Example 3: Final Product Questionnaire

After 2 weeks of product usage as described in Example 2, patients completed a final self-assessment questionnaire regarding perceptions of product performance. Outcomes for product measures are shown in Table 8. Subjects rated statements using completely agree, agree, neutral, disagree, and completely disagree.

TABLE 8

| | Agree/Completely |
|---|---|
| Product Measure | Agree % Agreement |
| Provides instant soothing relief | 97 |
| Soothes and relieves my itch | 94 |
| Provides cooling relief | 91 |
| Instantly cools and soothes my dry itchy skin | 88 |
| My dry skin area has a more even tone | 90.4 |

A statistical significantly greater proportion of subjects selected favorable responses (strongly agree and agree) compared to unfavorable responses (strongly disagree and disagree) to the statements, as listed in Table 8. Cooling, soothing itch, moisture relief with these levels of positive agreement are surprising. In particular, 91% of subjects agreed or completely agreed with the statement that the tested product provided cooling relief. This is surprising given that isododecane is not a known cooling agent, and yet the formula was well-tolerated by those having eczema. Also given the results shown in Example 5 below, the cooling effect of isosodecane is especially surprising, given that it does not provide the cooling effect at the same rate in subjects who don't have eczema versus those who do. This shows the promise of isododecane as a gentle cooling agent suitable for those with eczema.

### Example 4 Thermal Imaging Cooling Study

Test composition, prepared as described above, and D.I. water were self-applied by the subject separately on the volar forearm. The cooling effect of the test composition was compared with D.I. water as the control. A FLIR thermal camera (Teledyne FLIR, Thousand Oaks, CA) was used to capture the skin surface temperature at baseline, immediately post-application, 5 min post application, 10 min post application, and 15 min post application. The displayed temperature range was adjusted to accommodate subject's baseline skin surface temperature.

For each subject two circular sites with about 1″ diameter were marked on the forearm of their choice. The two sites had about 3″ spacing between them. Subject for 15 min to acclimate with the ambient temperature before their baseline thermal images were taken.

Subject was then instructed to apply both the treatment and control as they normally would with personal care products at the designated sites. The subjects were allowed to spread and rub in the product larger than the marked site, but were instructed to avoid overlapping the products on their skin. The experiment was repeated on 5 subjects.

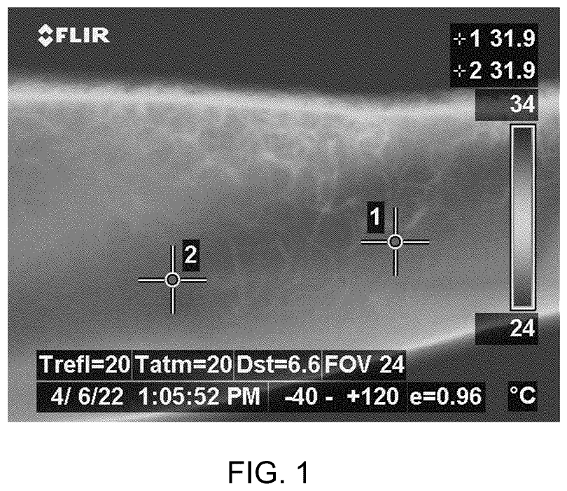

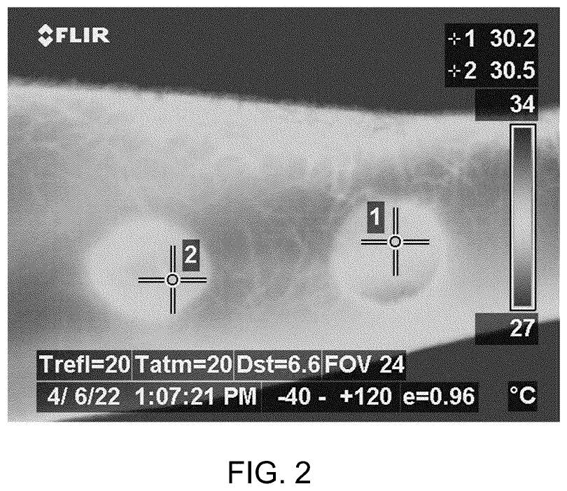

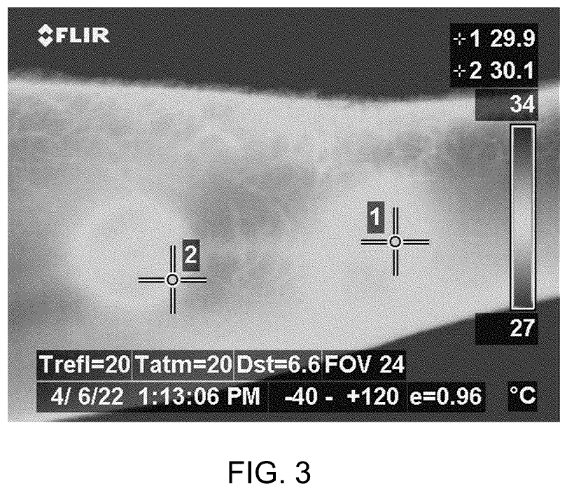

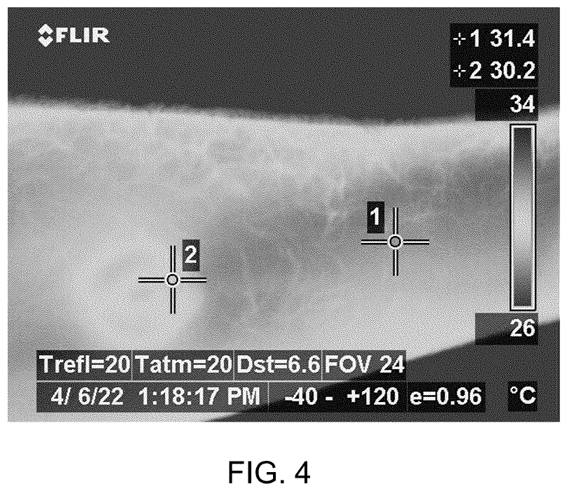

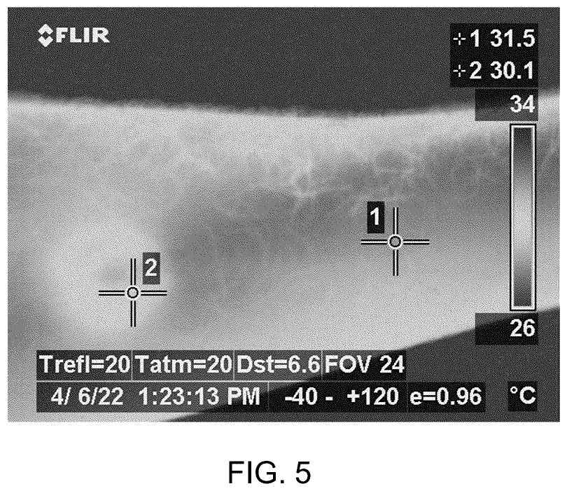

Representative thermal images in grayscale are shown in FIGS. 1-5 for baseline, immediately post-application, 5, 10, and 15 minutes post-application, respectively. The spot labeled 1 was the control while the spot labeled 2 was the test composition. The light gray circular spots marked by 1 and 2 show the cooling effect at 5-10 minutes, however at 15 minutes only spot 2, the test composition, continues to show the cooling effect. There was variation in the duration of the cooling effect for both the test composition and the control, in general the test composition had longer lasting cooling than the control.

### Example 5: Topical Cream and Gel Cream with Isododecane Clinical Study in Individuals not Having Eczema (Comparative)

A topical gel cream composition (Example 5-1) was made having the following ingredients, as shown in Table 9.

TABLE 9

| Ingredient | Weight % |
|---|---|
| Purified Water | Q.S. |
| Colloidal Oat Flour | 1.00 |
| Carbomer (Carbomer Ultrez 10 Polymer)¹ | 0.40 |
| Citric Acid Anhydrous USP | 0.60 |
| Lidocaine USP | 4.00 |
| Glycerin | 14.00 |
| Sodium Polyacrylate² | 0.75 |
| Isopropyl Palmitate³ | 5.00 |
| Cetearyl Olivate; Sorbitan Olivate⁴ | 0.50 |
| Cetyl Alcohol⁵ | 1.00 |
| Euphorbia Cerifera (Candelilla) Wax; Hydrogenated | 2.00 |
| Vegetable Oil; Vegetable Oil⁶ | |
| Isododecane⁷ | 4.00 |
| Zingiber Officinale (Ginger) Root Extract | 0.20 |
| Caprylyl Glycol⁸ | 0.26 |
| 1,2-Hexanediol; Caprylyl Glycol; Tropolone⁹ | 0.50 |
| Chlorphenesin¹⁰ | 0.30 |

¹Commercially available as Ultrez 10 Polymer from the Lubrizol Corporation
²Commercially available as Cosmedia ® SP from the UL Prospector Corporation
³Commercially available as Radia 7732 from the Oleon Corporation
⁴Commercially available as Olivem 1000 from the Hallstar BPC Corporation
⁵Commercially available as Vegarol 1698 from the UL Prospector Corporation
⁶Commercially available as Cegesoft VP from the UL Prospector Corporation
⁷Commercially available as Permethyl 99A from the Presperse Corporation
⁸Commercially available as Hydrolite CG from the Symrise Corporation
⁹Commercially available as Symdiol 68T from the Symrise Corporation
¹⁰Commercially available as Germazide C from the BASF Corporation

The following mixing procedure may be used to prepare Example 5-1:
a) The purified water is added to a suitable vessel and the colloidal oat flower is added while mixing
b) The citric acid and lidocaine USP are added while mixing and mixed until dissolved
c) The carbomer is added while mixing slowly until all clumps are dissolved
d) The mixture is heated until 75-80° C.
e) The caprylyl glycol and chlorphenesin are added while mixing and mixed until uniform.
f) The sodium polyacrylate is then added slowly while mixing at 75-80° C. until uniform
g) The oil phase ingredients are then added while mixing (7% of the glycerin, isopropyl palmitate, cetearyl olivate; sorbitan olivate, and cetyl alcohol)
h) The mixture is mixed for 10 minutes and then homogenized for 3 minutes at 5000 RPM
i) The mixture is cooled to 25° C. and additional 7% of glycerin and the isododecane is added and mixed
j) The Zingiber officinale root extract, 1,2-Hexanediol; Caprylyl Glycol; Tropolone are added and mixed until homogeneous.
k) At or below 35° C., the pH is adjusted to 6.5 using Sodium Hydroxide Solution or Citric Acid Solution and mixed.
l) The batch is brought to volume with water to 95-100% of theoretical batch weight.

A topical cream composition (Example 5-2) was made having the following ingredients, as shown in Table 10.

TABLE 10

| Ingredient | Weight % |
|---|---|
| Purified Water | Q.S. |
| Colloidal Oat Flour | 1.00 |
| Carbomer (Carbomer Ultrez 10 Polymer)¹ | 0.40 |
| Citric Acid Anhydrous | 0.60 |
| Lidocaine USP | 4.00 |
| Glycerine | 12.00 |
| Sodium Polyacrylate² | 0.60 |
| Isopropyl Palmitate³ | 5.00 |
| Cetearyl Olivate; Sorbitan Olivate⁴ | 0.50 |
| Cetyl Alcohol⁵ | 3.00 |
| Euphorbia Cerifera (Candelilla) Wax; Hydrogenated | 3.00 |
| Vegetable Oil; Vegetable Oil⁶ | |
| Isododecane⁷ | 8.00 |
| Fragrance | 0.50 |
| Acrylates Copolymer⁸ | 2.00 |
| Caprylyl Glycol⁹ | 0.26 |
| 1,2-Hexanediol; Caprylyl Glycol; Tropolone¹⁰ | 0.50 |
| Chlorphenesin¹¹ | 0.30 |

¹Commercially available as Ultrez 10 Polymer from the Lubrizol Corporation
²Commercially available as Cosmedia ® SP from the UL Prospector Corporation
³Commercially available as Radia 7732 from the Oleon Corporation
⁴Commercially available as Olivem 1000 from the Hallstar BPC Corporation
⁵Commercially available as Vegarol 1698 from the UL Prospector Corporation
⁶Commercially available as Cegesoft BP from the UL Prospector Corporation
⁷Commercially available as Permethyl 99A from the Presperse Corporation
⁸Commercially available as Carbopol Aqua SF-1 Polymer from the Lubrizol Corporation
⁹Commercially available as Hydrolite CG from the Symrise Corporation
¹⁰Commercially available as Symdiol 68T from the Symrise Corporation
¹¹Commercially available as Germazide C from the BASF Corporation

The following mixing procedure may be used to prepare the cream of this example:
a) The purified water is added to a suitable vessel and the colloidal oat flower is added while mixing
b) The citric acid and lidocaine USP are added while mixing and mixed until dissolved
c) The carbomer is added while mixing slowly until all clumps are dissolved
d) The mixture is heated until 75-80° C.
e) The caprylyl glycol and chlorphenesin are added while mixing and mixed until uniform.
f) The sodium polyacrylate is then added slowly while mixing at 75-80° C. until uniform
g) The oil phase ingredients are then added while mixing (7% of glycerin, isopropyl palmitate, cetearyl olivate; sorbitan olivate, and cetyl alcohol)
h) The mixture is mixed for 10 minutes and then homogenized for 3 minutes at 5000 RPM
i) The mixture is cooled to 25° C. and the isododecane is added and mixed
j) The fragrance, 1,2-hexanediol; caprylyl glycol; tropolone are added and mixed until homogeneous.
k) At or below 35° C., the pH is adjusted to 6.5 using sodium hydroxide solution or citric acid solution and mixed.
l) In a different vessel 5% glycerin and acrylates Copolymer are added and mixed until uniform to create the acrylates copolymer premix.
m) The acrylates copolymer premix is added to the mixture from Step 11 and mixed until homogenous.

### Human Use Study

### Study Protocol and Objective:

A home use test (abbreviated as “HUT”) was conducted where 253 consumers (also referred to as “subjects,” “patients” or “users”) self-administered the formulas in Examples 5-1 and 5-2 (also referred to in this example as “formula(s)” or “product(s)”) as needed for a period of two weeks. These consumers were between 30 and 55 years old, who regularly or occasionally experience minor aches and pains of the muscles or joints, and who had a range of skin tones and types with a sub-set having self-reported sensitive skin, dry skin, and/or eczema.

The consumers were instructed to use the product within the first two days of receiving it and then at least once a week as needed after that, over the following two weeks. The consumers were further instructed to abstain from using other pain-relieving treatments during the two-week period. The consumers provided feedback via an online survey at three time points: 1) after first use, 2) after one week of use, and 3) after two weeks of use.

Example 5-1 may be referred to as a “soothing gel cream” incorporating, for example, ginger and Example 5-2 may be referred to as a “calming cream” incorporating a suitable calming fragrance, for example, lavender.

The objective of the human use study was to generate support for potential consumer perceptual claims centered around a consumers' experience with the product. For example, an objective was to understand the consumers' experience with regard to product absorption, discreetness and feel on the skin.

With regard to feel, consumers provided feedback on whether the product was perceived to be gentle and whether the product made their skin feel moisturized and/or silky.

A “test statement” may be a question that is answered or a statement that is agreed or disagreed with at least once by the consumer during the duration of the HUT. According to an embodiment, the following test statements may relate to the consumers' experience with regard to product absorption: is fast absorbing, instantly absorbs into the skin, is non-greasy, and/or doesn't leave my skin shiny. According to an embodiment the following test statements may relate to the consumers' experience with regard to “discreetness:” is lightweight, does not have a harsh smell/odor, has no unpleasant smell/odor, is non-sticky, is non-greasy, doesn't leave my skin shiny, has a pleasant smell. According to an embodiment, the following test statements may relate to the consumers' experience with regard to product feel: is lightweight, is non-sticky, is non-greasy, instantly moisturizes, leaves my skin feeling silky, instantly cools, feels like it is working right away and/or instantly numbs. The test statements of instantly cools, feels like it is working right away and/or instantly numbs may indicate pain relief.

### Summary of the Results

The top line data is presented in Table 11, and shows the reactions as described by consumer, collated as a percentage of users in agreement with the test statement. Total results, as well as results for each of Example 5-1 and Example 5-2 are shown.

TABLE 11
Human Use Study Results

| | Percentage (%) of Consumers in Agreement | | |
|---|---|---|---|
| | | Example | Example |
| | Total | 5-1 | 5-2 |
| Test Statement | (n = 249) | (n = 127) | (n = 122) |
| is easy to apply | 98 | 98 | 98 |
| is lightweight | 95 | 95 | 94 |
| does not have a | 94 | 94 | 93 |
| harsh smell/odor | | | |
| has no unpleasant | 92 | 94 | 90 |
| smell/odor | | | |
| is non-sticky | 92 | 89 | 94 |
| is fast absorbing | 90 | 90 | 91 |
| instantly absorbs | 87 | 87 | 88 |
| into the skin | | | |
| is non-greasy | 87 | 87 | 87 |
| instantly moisturizes | 83 | 79 | 88 |
| doesn't leave my | 82 | 86 | 78 |
| skin shiny | | | |
| has a pleasant smell | 79 | N/A | 79 |
| leaves my skin feeling silky | 73 | 72 | 73 |
| instantly cools | 63 | 61 | 64 |
| feels like it's | 62 | 61 | 64 |
| working right away | | | |
| instantly numbs | 53 | 50 | 55 |

* Not Applicable: Formula was fragrance free.

As can be seen from the above, the percentage of subjects reporting a cooling effect is 61% for 5-1 and 64% for 5-2. As described above, this study included subjects who did not have eczema. Thus the presence of isododecane does not necessarily provide a cooling effect that is perceived at the same rate as in those having eczema, even when present in amounts as high as 4% and 8%, which are much higher than the 2% present in the formulation of Example 1.

### Example 6: Microbiome Clinical Study

A clinical study was conducted according to the methods described in Example 2. At Visit 1 (baseline), Visit 2 (day 3), Visit 3 (day 7), and Visit 4 (day 14), surface skin microbiome was collected from each subject's target lesion. Swabbing was conducted within a 4 cm×4 cm area of skin on the identified target lesion. A sterile swab was dipped into an aliquot of 0.85% sterile saline and wrung of excess liquid by pressing it against the inner wall of the saline tube. The lateral edge of the swab was rubbed across the entire defined area while being rotated between the thumb and forefinger for 60 seconds. More specifically, the rotating swab was rubbed back and forth in a crosswise manner in the defined area in the same fashion for each subject to maintain consistency. The head of each swab was placed into a sterile microcentrifuge tube and aseptically cut from the handle before the lid was closed. The tube was labeled with the protocol ID, subject ID, and visit number. Post-baseline swabs were performed on the same location as baseline.

A control swab was taken on each day a subject was sampled, after the subject swabs were collected. A sterile swab was dipped into an aliquot of 0.85% sterile saline and wrung of excess liquid by pressing it against the inner wall of the saline tube. Without touching the polyester tip to any surface, the head of the swab was placed into a sterile microcentrifuge tube and aseptically cut from the handle before the lid was closed. The tube was labeled with the protocol number, date, and “Micro Control” (or equivalent identification).

Swabs from each subject and control swabs were frozen at −20° C. until shipment on dry ice to a Sponsor-appointed external laboratory (CosmosID Inc., Germantown, MD) for DNA extraction and analysis to determine the effect of the IP on the skin microflora. In total, 154 16S samples were utilized in the analysis. All figures were generated using 16S OUT analysis from the CosmosID Hub, and adjusted by 16S qPCR abundances with control abundances subtracted from all other samples.

Taxonomic comparative statistical analysis was performed by the external laboratory, including qPCR Abundance, Relative Abundance, Alpha Diversity, and Beta Diversity. qPCR Abundance is the mean abundance per group and scaled abundance for each sample. Relative abundance is the mean abundance of top bacterial species per group. Alpha diversity is the number and abundance of taxa and the differences per group. Beta diversity is the dissimilarity of the overall microbial communities and shows if the cohorts are significantly different.

Staphylococcus aureus (S. aureus) and Staphylococcus epidermidis (S. epidermidis) were determined by Sample and by Cohort. Mean abundance per group and scaled abundance for each sample were calculated. Stacked Bar figures were generated using phylum, genus, and species-level filtered matrices from CosmosID. Stacked bar figures for each group were generated using the R package ggpubr.

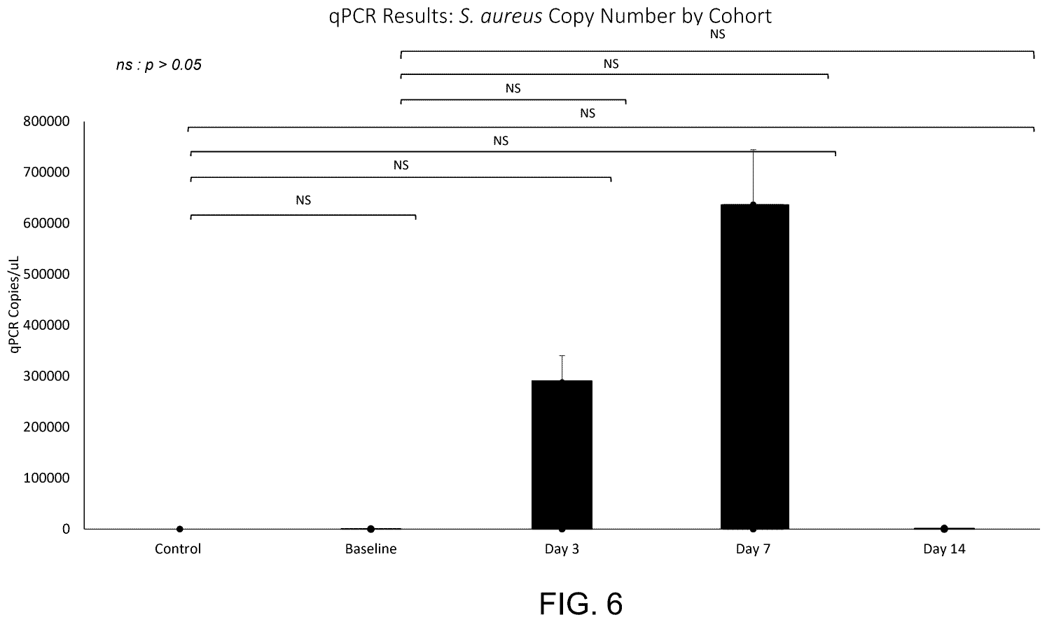

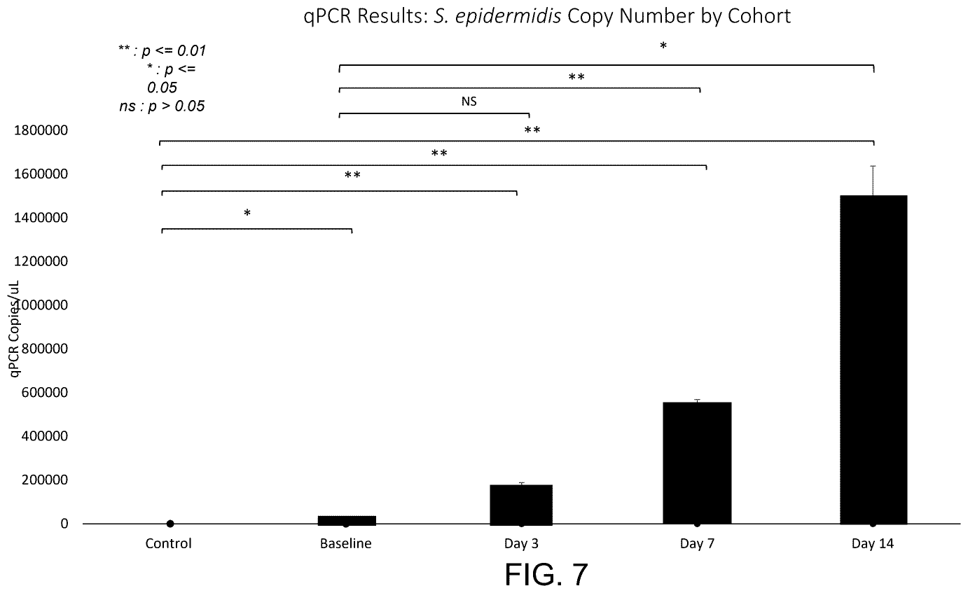

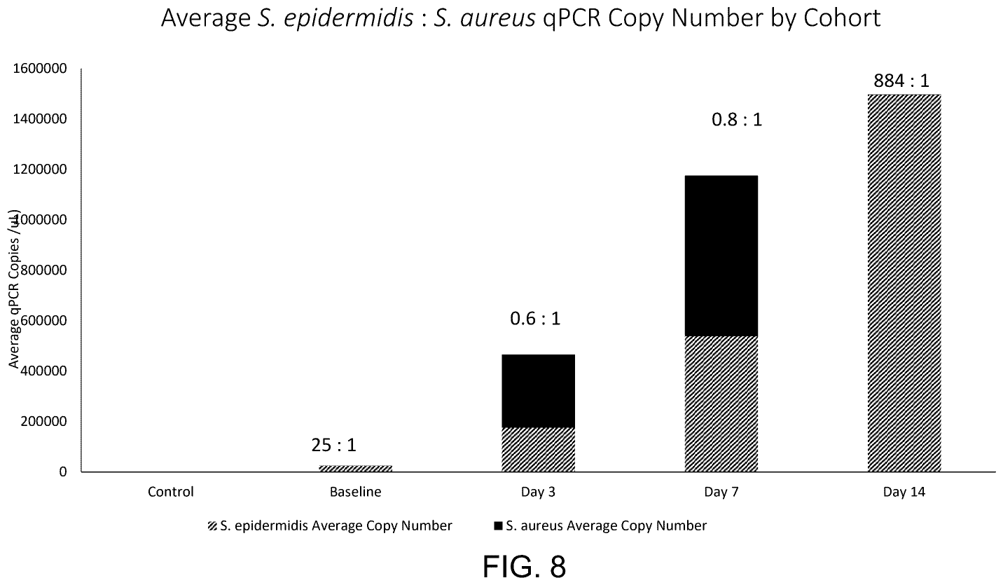

Staphylococcus aureus and Staphylococcus epidermidis copy number by cohort are shown in FIGS. 6 and 7 respectively, while the ratio of average Staphylococcus epidermidis to average Staphylococcus aureus copy number by cohort are shown in FIG. 8. There was no significant change in S. qPCR copy number between baseline and day 14. However, there was a significant increase in S. epidermidis qPCR copy number between baseline and day 14. As a result, the ratio of S. epidermidis to S. aureus increased between baseline and day 14. These results show the samples were unexpectedly augment commensal S. epidermidis by the test composition.

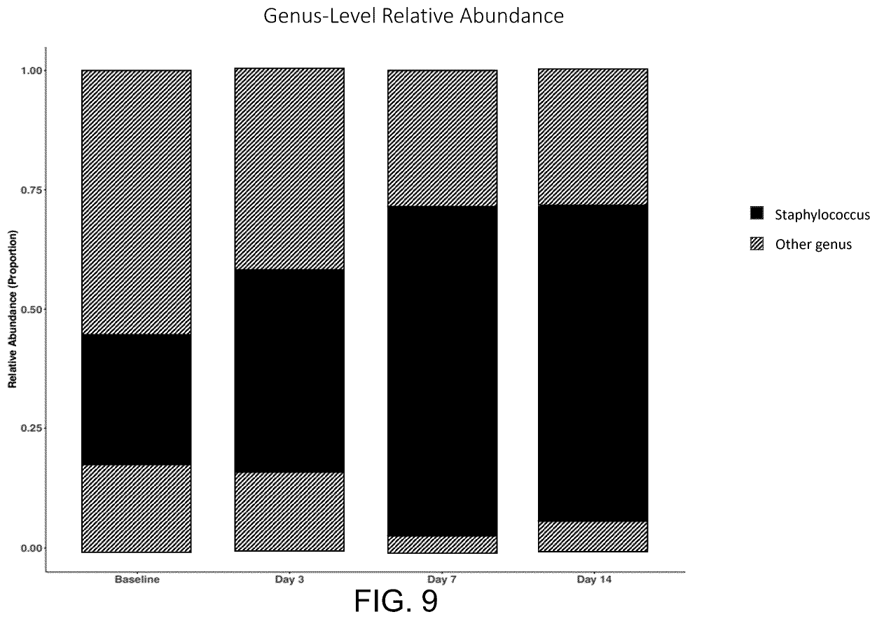

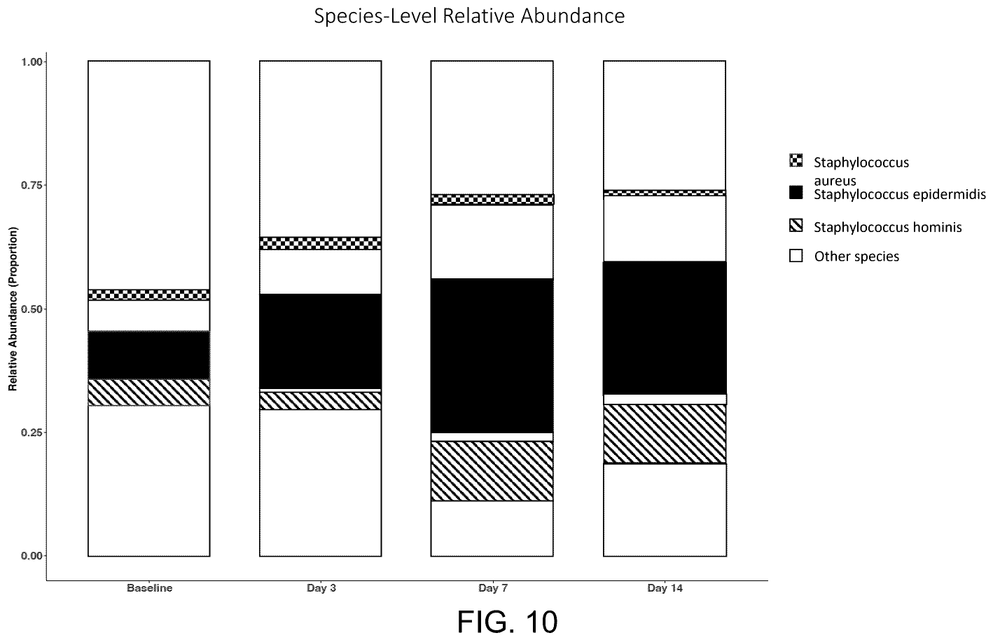

Genus level relative abundance and species level relative abundance are shown in FIGS. 9 and 10, respectively. There was a visual increase in Staphylococcus genus between baseline and day 14 relative to other genus. At the species level, there was an increase in S. epidermidis between baseline and day 14; and a slight decrease in S. aureus between baseline and day 14, again relative to the level of other species present. An increase was observed in other commensals, including an unexpected increase in S. hominis relative abundance between baseline and Day 14.

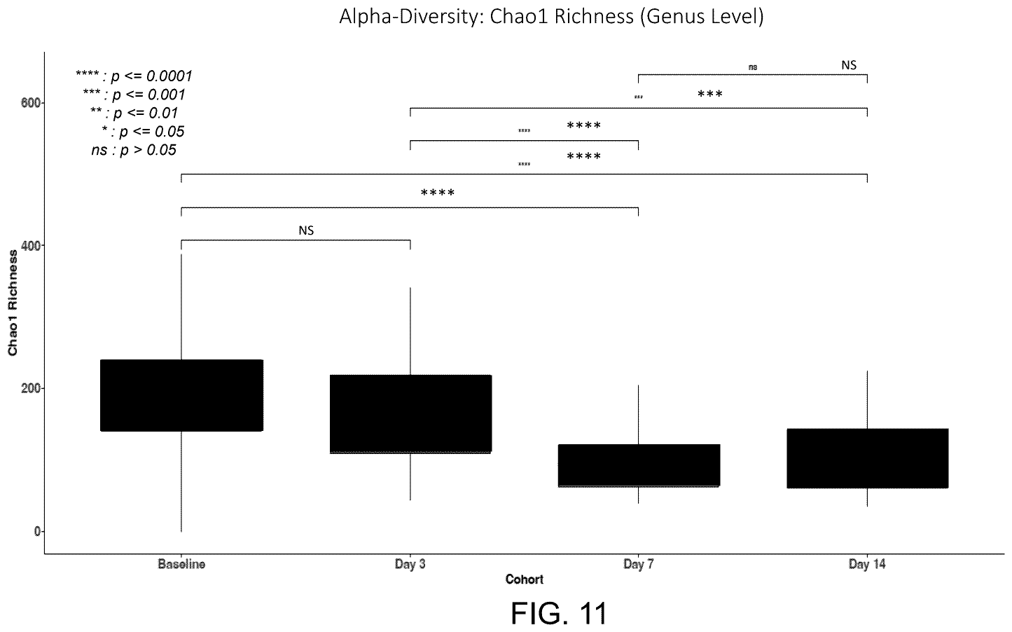

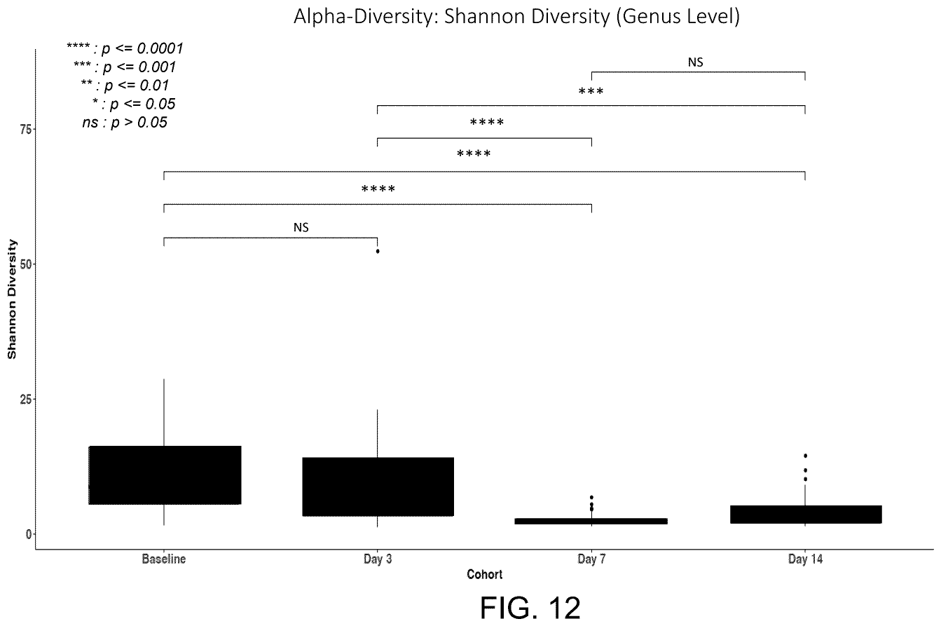

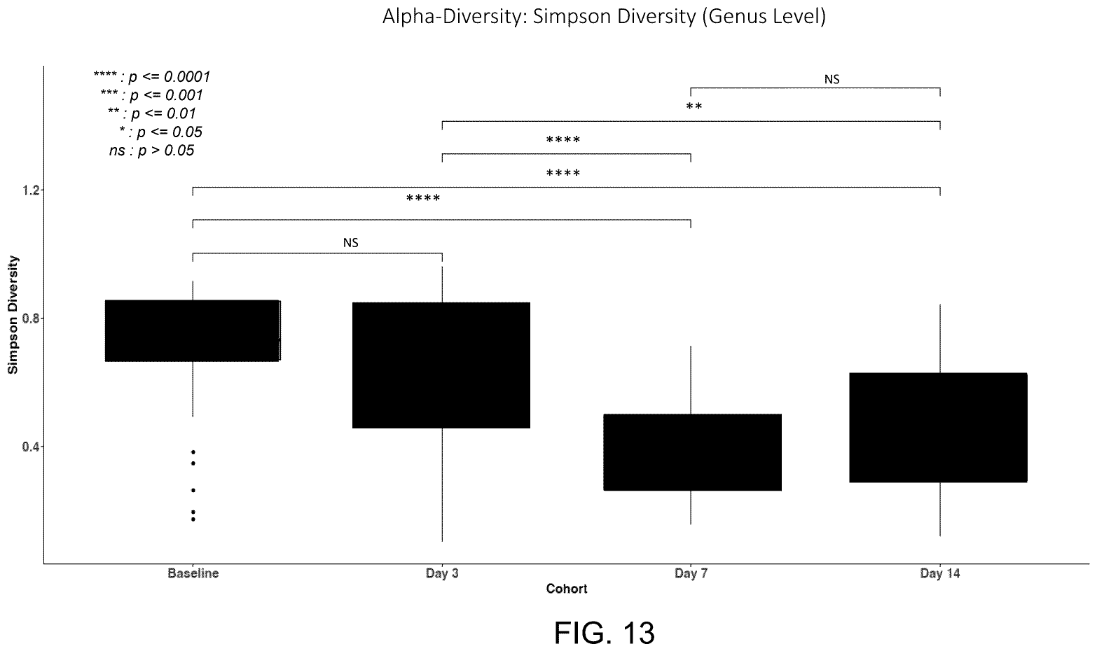

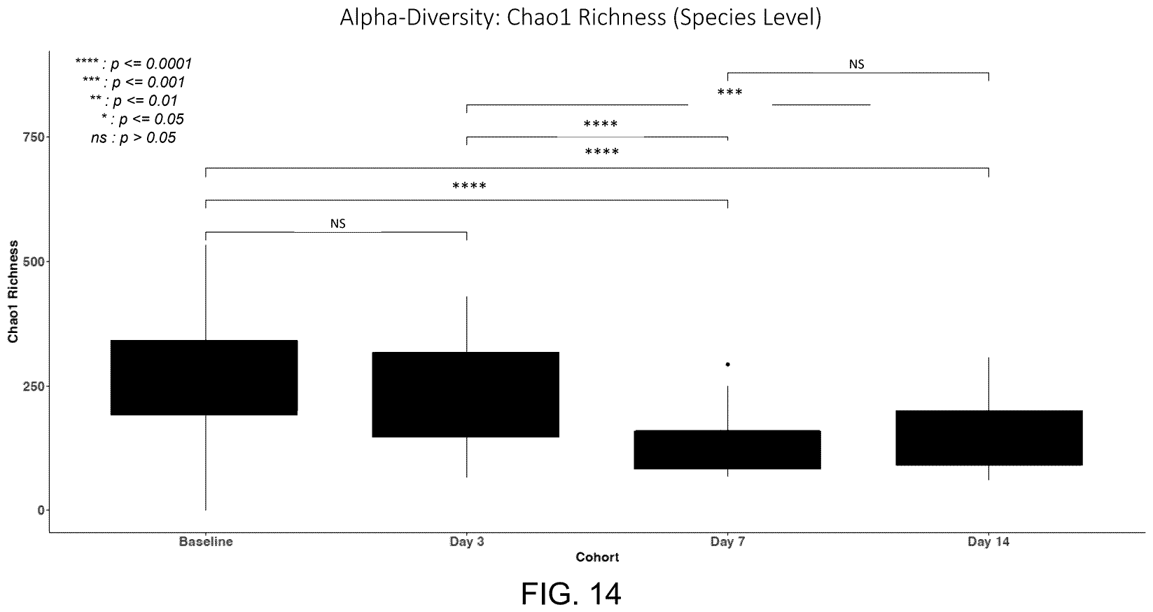

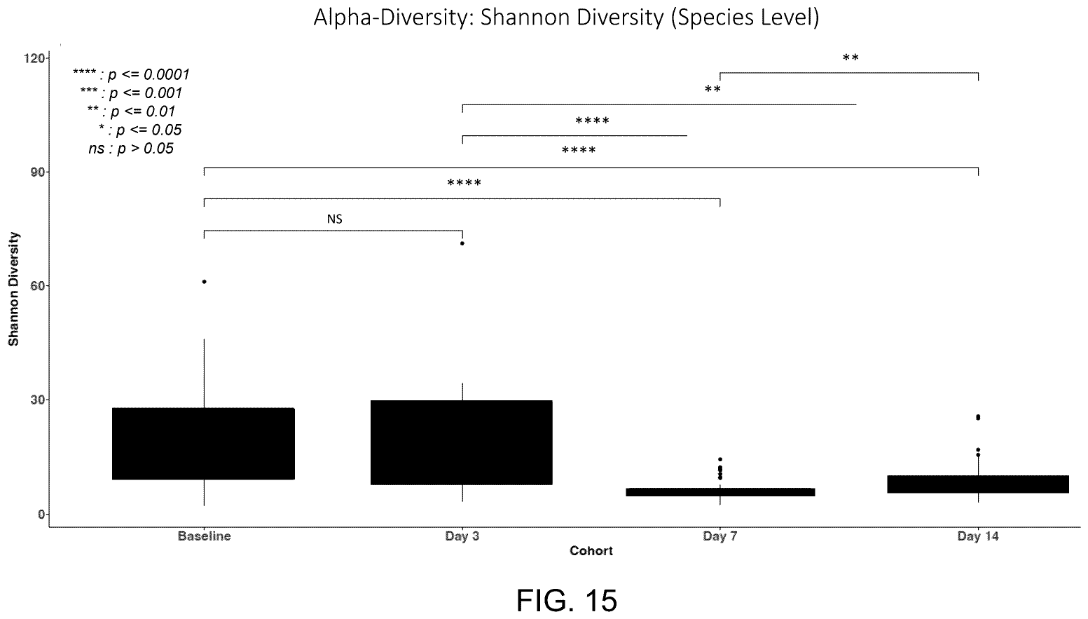

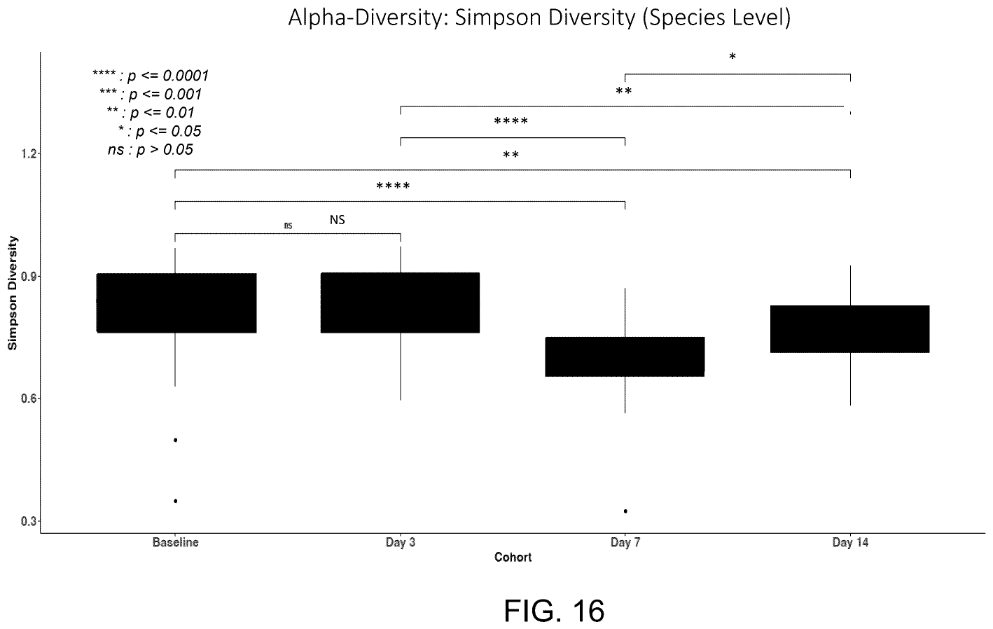

Alpha diversity boxplots were calculated from the genus and species level abundance score matrices respectively, from CosmosID analysis. Chao, Simpson, and Shannon alpha diversity metrics were calculated in R using the R package Vegan. Wilcoxon Rank-Sum tests were performed between groups using the R package ggsignif. Boxplots with overlaid significance in p-value format were generated using the R package ggplot2. A significant decrease across all alpha diversity indices (Chao1 FIGS. 11 and 14, Shannon FIGS. 12 and 15, Simpson FIGS. 13 and 16) between baseline and day 14 at both the genus and species level. Alpha diversity between day 7 and day 14 showed trending as a potential increase: significant increase between day 7 and day 14 observed in species-level Shannon Diversity and Simpson Diversity analyses. Generally, it is thought that increasing diversity is preferred, and thus seeing an increase is surprising given the very positive clinical results of Example above.

Beta Diversity Principal Coordinate Analyses were calculated from phylum, genus, and species-level filtered matrices from CosmosID. Bray-Curtis diversity was calculated in R using the R package Vegan with the functions vegdist, and PCoA tables were generated using ape's function pcoa. PERMANOVA tests for each distance matrix were generated using vegan's function adonis2. Plots were visualized using the R package ggpubr. The PERMANOVA results are shown in Table 12 and the Pairwise PERMANOVA results are shown in Table 13 below. a significant change in beta diversity between baseline and day 14 was observed which indicated significantly different microbial communities.

TABLE 12
PERMANOVA

| | | | | | p- | |
|---|---|---|---|---|---|---|
| Pairs | Df | SumsofSqs | R2 | F. Model | value | Signif |
| Cohort | 3 | 4.510374 | 0.08050339 | 3.881454 | 0.001 | *** |
| Resid- | 133 | 51.516758 | 0.91949661 | NA | NA | NA |
| ual | | | | | | |
| Total | 136 | 56.027132 | 1.00000000 | NA | NA | NA |

***p <= 0.001

TABLE 13
Pairwise PERMANOVA

| | | | | | p- | |
|---|---|---|---|---|---|---|
| Pairs | Df | SumsofSqs | R2 | F. Model | value | Signif |
| Baseline | 1 | 0.5145805 | 0.01904598 | 1.300857 | 0.164 | NS |
| vs Day 3 | | | | | | |
| Baseline | 1 | 2.5221164 | 0.08899577 | 6.642903 | 0.001 | *** |
| vs Day 7 | | | | | | |
| Baseline | 2 | 2.3654925 | 0.07872146 | 2.947958 | 0.001 | *** |
| vs Day 4 | | | | | | |

***p <= 0.001

### Example 7 In Vitro Bacteria Competition Assay

In vitro bacteria competition assay evaluated Staphylococcus epidermidis (ATCC 12228, skin health associated strain) and Staphylococcus aureus (ATCC 6538, human lesion isolate) co-culture in the presence of test materials. Percentages are w/v % in liquid medium (Nutrient Broth, Culture Media and Supplies Inc.) To mimic the microenvironment of the skin surface, an aerobic, co-culture system of S. epidermidis and S. aureus was established. 50 μL S. epidermidis and 50 μL S. aureus (a 1:1 ratio) were inoculated (50 μL at approximately 5.0×10⁶ CFU/mL each) in Nutrient Broth and in the presence of the test materials as specified in Table 14 below.

TABLE 14

| | Amount of | Nutrient |
|---|---|---|
| Treatment | test material | broth (mL) |
| Untreated | | 9.9 |
| 2% Colloidal | 0.2 g Colloidal | 9.7 |
| oat flour | oat flour¹ | |
| 1% Oat extract | 0.1 g Oat extract² | 9.8 |
| 0.5% Glycerin | 0.05 g glycerin³ | 9.95 |
| 5% Oat oil | 0.05 g Oat oil⁴ | 9.85 |
| 1.1% Lactic acid | 0.11 g lactic acid^{5, 6} | 9.79 |
| 2% Colloidal Oat | 0.1 g Oat Extract + | 9.6 |
| Flour + 1% Oat | 0.2 g Colloidal | |
| Extract | Oat Flour | |
| 2% Colloidal Oat | 0.2 g Colloidal Oat | 9.55 |
| Flour + 1% Oat | Flour + 0.1 g Oat | |
| Extract + 0.5% | Extract + 0.05 g | |
| Oat Oil | Oat Oil | |
| 2% Colloidal Oat | 0.2 g Colloidal Oat | 9.44 |
| Flour + 1% Oat | Flour + 0.1 g Oat | |
| Extract + 0.5% | Extract + 0.05 g | |
| Oat Oil + 1.1% | Oat Oil + 0.11 g | |
| Lactic Acid | Lactic Acid⁶ | |

¹100% Colloidal Oatmeal
²0.55% Avena Sativa Kernal Extract, 49.625% Glycerin, 49.625% water, 0.2 potassium sorbate
³100% Glycerin
⁴100% Avena Sativa (Oat) Kernel Oil
⁵90% Lactic acid, 10% water
⁶pH-adjusted to 4.5 with 10% NaOH

Test groups were incubated aerobically for 24 hours (n=3 per test group) After 24 hours, each test group was plated onto agar and incubated aerobically for 24 hours shaking at 200 rpm. After incubation, colonies were counted, and CFU/mL calculated. S. epidermidis and S. aureus colonies were differentiated on agar (Tryptic Soy Agar+5% sheep's blood, BD Diagnostics) based on representative morphology.

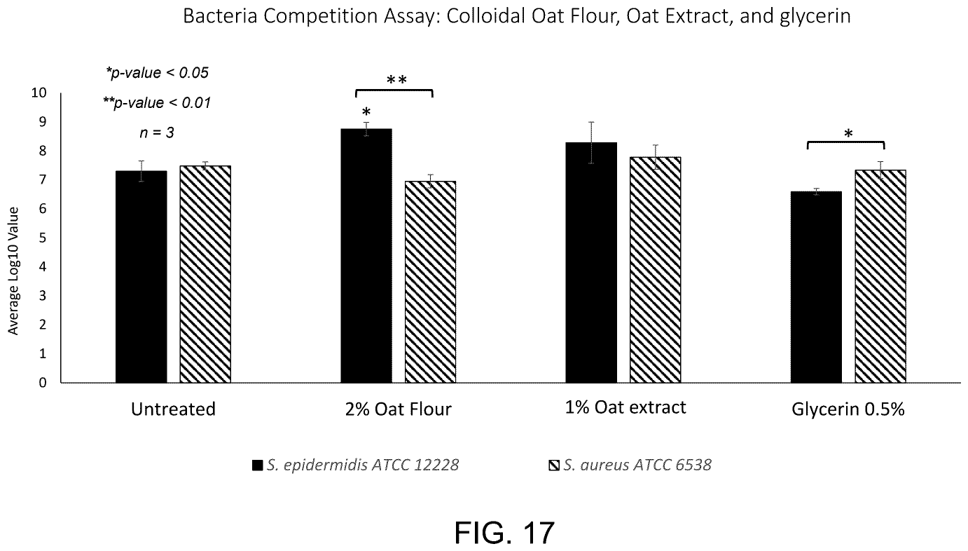

FIG. 17 depicts the bacteria competition assay results for colloidal oat flour, oat extract, and glycerin (a component in the oat extract). 2% oat flour significantly promoted the growth of S. epidermidis when compared to untreated. The level of S. epidermidis was significantly greater than that of S. aureus when cultured in the presence of 2% oat flour.

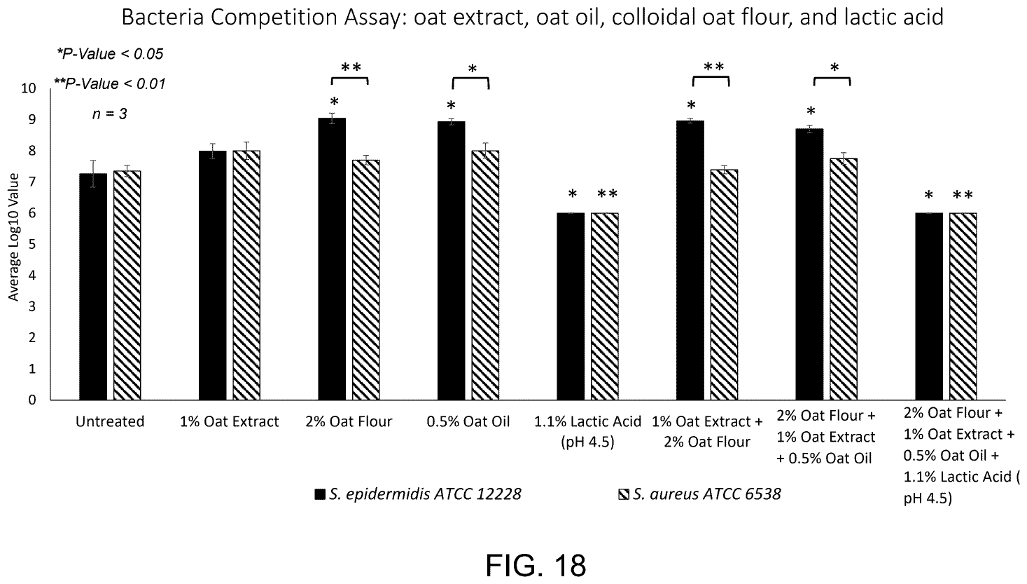

FIG. 18 shows the bacteria competition assay results for oat extract, oat flour, oat oil, lactic acid, and combinations as described in Table 15. 2% oat flour and 0.5% oat oil each significantly promoted the growth of S. epidermidis over S. aureus and versus untreated. 1% Oat Extract+2% Oat Flour significantly promoted the growth of S. epidermidis over S. aureus and versus untreated. 2% Colloidal Oat Flour+1% Oat Extract+0.5% Oat Oil also significantly promoted the growth of S. epidermidis over S. aureus and versus untreated.

TABLE 15

| | |
|---|---|
| Test | Bacteria Competition Assay |

| Material(s) | S. epidermidis | S. aureus |
|---|---|---|
| Oat Flour | Significantly | No |
| (2% ) | augmented | significant |
| | | impact |
| Oat Extract | No | No |
| (1%) | significant | significant |
| | impact | impact |
| Oat Oil | Significantly | No |
| (0.5%) | augmented | significant |
| | | impact |
| 2% Oat | Significantly | No |
| Flour + 1% | augmented | significant |
| Oat Extract + | | impact |
| 0.5% Oat Oil | | |
| 2% Oat | Significantly | No |
| Flour + 1% | augmented | significant |
| Oat Extract | | impact |
| Lactic Acid | Significantly | Significantly |
| (1.1%) | inhibited | inhibited |
| 2% Oat | Significantly | Significantly |
| Flour + 1% | inhibited | inhibited |
| Oat Extract + | | |
| 0.5% Oat | | |
| Oil + 1.1% | | |
| Lactic Acid | | |

The resulting prebiotic activity (significantly augmented commensal S. epidermis) of Oat Flour in Ex. 6 is consistent with that of the Bacterial Rate of Growth Assay results shown in Ex.7. Interestingly, the presence of lactic acid inhibited S. epidermis, low populations of which are associated with atopic dermatitis. These results were seen for lactic acid both alone and in combination with the three oat components, which shows augmentation for S. epidermis. Such results are thus surprising given the very positive clinical results of Example above.

1. A composition suitable for skin afflicted with eczema, the composition comprising:
a. an alpha hydroxy acid;
b. a volatile emollient; and
c. a processed oat ingredient.

2. The composition of claim 1, wherein the alpha hydroxy acid is selected from the group consisting of glycolic acid, malic acid, tartaric acid, pyuric acid, mandelic acid, lactic acid, and combinations thereof.

3. The composition of claim 1, wherein the alpha hydroxy acid is present in an amount ranging from about 0.01 wt. % to about 5 wt. % by total weight of the composition.

4. The composition of claim 1, wherein the volatile emollient is selected from the group consisting of cyclopentasiloxane, dimethicone, isoparaffin cuts (C9-C16), ethyl trisiloxane, trisiloxane and caprylyl methicone, isododecane, and combinations thereof.

5. The composition of claim 1, wherein the volatile emollient is present in an amount ranging from about 0.01 wt. % to about 12 wt. % by total weight of the composition.

6. The composition of claim 1, wherein the processed oat ingredient is selected from the group consisting of oat extract, colloidal oatmeal, oat oil, fermented oat, avenanthramides, beta-glucan, modified oat grain material, and combinations thereof.

7. The composition of claim 1, wherein the processed oat ingredient is present in an amount ranging from about 0.01 wt. % to about 30 wt. % by total weight of the composition.

8. The composition of claim 1, further comprising a dermatologically-acceptable ingredient selected from the group consisting of viscosity increasers, humectants, skin conditioners, emulsifiers and combinations thereof.

9. The composition of claim 1, wherein the composition has a pH of from about 4.5 to about 5.5.

10. The composition of claim 1, wherein the composition is in the form of a lotion, gel or cream product.

11. A method of treating eczema, the method comprising topically applying to skin afflicted with eczema the composition of any of claim 1.

12. A composition suitable for skin afflicted with eczema, the composition comprising:
a. lactic acid;
b. isododecane; and
c. a processed oat ingredient selected from the group consisting of oat extract, colloidal oatmeal, oat oil and combinations thereof.

13. The composition of claim 12, wherein the composition comprises:
a. from about 0.01 wt. % to about 3 wt. % lactic acid;
b. from about 0.01 wt. % to about 4 wt. % isododecane; and
c. from about 0.01 wt. % to about 6 wt. % combined of oat extract, colloidal oatmeal, and oat oil.

14. A method of treating eczema, the method comprising applying to skin afflicted with eczema the composition of any of claim 12.

15. The method of claim 14, wherein the skin afflicted with eczema comprises mild to moderate eczema.
